(19) United States
(12) Patent Application Publication
(10) Pub. No.: US 2011/0318267 A1
(43) Pub. Date: Dec. 29, 2011

(54) PH-RESPONSIVE NANOSTRUCTURES

(75) Inventors: Debra Auguste, Arlington, MA (US); Jin-Oh You, Cambridge, MA (US)

(73) Assignee: PRESIDENT AND FELLOWS OF HARVARD COLLEGE, CAMBRIGE (MA)

(21) Appl. No.: 12/864,265

(22) PCT Filed: Jan. 23, 2009
(86) PCT No.: PCT/US09/00435

§ 371 Date: Sep. 14, 2011

(30) Foreign Application Priority Data
Feb. 25, 2008 (US) 61062387

(51) Int. Cl. A61K 49/00; A61K 31/337; A61P 35/00; A61K 47/32; A61K 9/14; B82Y 5/00

(52) U.S. Cl. 424/9.1; 514/772.4; 424/400; 514/449; 977/773; 977/906

(57) ABSTRACT

The present invention relates to compositions comprising stimuli-responsive materials which can be used for delivery of agents. Materials are provided that can respond to changes in a surrounding environment, including changes in ion concentration. For example, one or more properties of the material may be affected by a change in pH and/or a change in the concentration of an ion. Such properties may include, for example, a dimension of the material (e.g., volume), the permeability of the material, and the like. In cases where the material comprises a therapeutic or diagnostic agent, the material may undergo a change in the rate of release of the agent upon exposure to certain conditions. The invention may also provide the use of said compositions for the manufacture of a medicament for delivering a therapeutic or diagnostic agent, in some cases, with enhanced selectivity and sensitivity.

### FIELD OF THE INVENTION

The present invention relates to compositions and methods comprising stimuli-responsive materials, and in some cases, for delivering a therapeutic or diagnostic agent.

### BACKGROUND OF THE INVENTION

Stimuli-sensitive materials that respond to stimuli such as temperature, pH, light, ionic strength, and electric or magnetic fields are attractive for use in drug delivery, diagnostics, and sensing. Much attention has been paid to the use of pH-responsive materials for oral drug delivery, gene delivery, and insulin delivery, where a physiological pH shift facilitates swelling. Because conventional pH-responsive functional groups provide insufficient sensitivity within the blood pH range (e.g., pH between 7.8 and 7.0), their use in systemic drug delivery systems has been limited.

Studies have been conducted relating to materials that are responsive to physiological conditions within the blood pH range. Some drug delivery systems have been designed to use pH as a mechanism for improving delivery of chemotherapeutics, wherein side effects such as nausea and hair loss may be reduced and the quality of life of patients may be improved.

While stimuli-responsive materials commonly employed in drug-delivery often swell at high pH values (e.g., basic pH values), this mechanism may not be useful for delivery of agents to the acidic environments associated with, for example, tumors. Tumors have been shown exhibit a lower extracellular pH relative to normal tissues, which can often result from a chaotic vasculature and deregulation of glycolysis. Previous stimuli-responsive materials, including particles, have been reported to swell under acidic conditions, but such materials typically require a large pH change, such as a pH change of at least 1.0 pH unit, which can limit their use in drug delivery applications. Accordingly, improved materials and methods are needed.

### SUMMARY OF THE INVENTION

The present invention relates to materials that change when at least one aspect of their surrounding environment changes, in a way such that they can serve as diagnostic and/or therapeutic agent delivery materials.

One aspect of the invention relates to methods. In one embodiment, the invention provides a method for delivering a therapeutic or diagnostic agent, which includes providing a pH-responsive nanostructure comprising a polymeric material and a therapeutic or diagnostic agent, exposing the pH-responsive nanostructure to a first set of conditions, wherein the pH-responsive nanostructure releases the therapeutic or diagnostic agent at a first release rate, and exposing the pH-responsive nanostructure to a second set of conditions, wherein the pH-responsive nanostructure releases the therapeutic or diagnostic agent at a second release rate that is at least about 20% greater than the first release rate, wherein the first and second sets of conditions comprise different pH values.

In another embodiment, a method of the invention involves providing a pH-responsive composition comprising a therapeutic or diagnostic agent, exposing the pH-responsive composition to a first set of conditions, wherein the pH-responsive composition releases the therapeutic or diagnostic agent at a first release rate, and exposing the pH-responsive composition to a second set of conditions, wherein the pH-responsive composition releases the therapeutic or diagnostic agent at a second release rate that is at least about 20% greater than the first release rate, wherein the first pH value differs from the second pH value by less than about 1.0 pH unit.

In another embodiment, a method of the invention involves providing a pH-responsive composition comprising a therapeutic or diagnostic agent, exposing the pH-responsive composition to a first set of conditions, wherein the pH-responsive composition releases the therapeutic or diagnostic agent at a first release rate, and exposing the pH-responsive composition to a second set of conditions, wherein the pH-responsive composition releases the therapeutic or diagnostic agent at a second release rate that is at least about 20% greater than the first release rate, wherein the first pH value is greater than the second pH value.

Another aspect of the invention relates to compositions. In one embodiment, the invention provides a composition for delivery of a therapeutic or diagnostic agent, including a pH-responsive nanostructure comprising a polymeric material, wherein the polymeric material comprises hydrophilic repeating units and pH-responsive repeating units, the ratio of hydrophilic repeating units to pH-responsive repeating units being at least about 1.0, and a therapeutic or diagnostic agent associated with the polymeric material, wherein the pH-responsive nanostructure has an average particle size of less than about 500 nm.

In another embodiment, the invention involves a composition for delivery of a therapeutic or diagnostic agent, including a pH-responsive nanostructure comprising a polymeric material, wherein the polymeric material comprises hydrophilic repeating units and pH-responsive repeating units, the ratio of hydrophilic repeating units to pH-responsive repeating units being at least about 1.0, and a therapeutic or diagnostic agent associated with the polymeric material, wherein, when exposed to a decrease in pH of about 1.0 pH unit or less, the pH-responsive nanostructure undergoes an increase in volume.

In another embodiment, the invention involves a composition for delivery of a therapeutic or diagnostic agent, including a plurality of pH-responsive nanostructures each comprising a base material and a therapeutic or diagnostic agent associated with the base material, wherein the therapeutic or diagnostic agent has a first release rate under a first set of physiologically-compatible conditions at a first pH, and a second release rate at least about 20% greater than the first rate under a second set of physiologically-compatible conditions at a second pH differing from the first pH by less than 1.0 pH unit.

### BRIEF DESCRIPTION OF THE DRAWINGS

FIG. 1 shows a TEM image of an ion-sensitive nanoparticle, according to one embodiment of the invention.

FIGS. 2A and 2B show graphs of the volume swelling ratios of two types of nanoparticles in one embodiment of the present invention, wherein the type types of nanoparticles differ by the ratio of repeating units.

FIG. 3 shows a graph of the swelling ratios of the nanoparticles in another embodiment.

FIG. 4 gives a table of the zeta potentials for nanoparticles in one embodiment of the present invention.

FIG. 5A show a graph of the controlled release of a substance from nanoparticles in some embodiments of the present invention, wherein the pH is varied.

FIG. 5B show a graph of the controlled release of a substance from the nanoparticles in some embodiments of the present invention, wherein the percent crosslinking agent within the nanoparticles is varied.

FIG. 6 shows the triggered release of a substance in other embodiments.

FIG. 7 shows a graph of the viable cells in certain examples of the present invention.

FIG. 8A shows a fluorescent image of the nucleus of cells comprising nanoparticles labeled with quantum dots and green fluorescence protein (GFP) expressed due to delivery of GFP plasmid by the nanoparticles, in a certain embodiment.

FIG. 8B shows a fluorescent image of GFP expressed due to delivery of GFP plasmid to a cell by the nanoparticles.

FIG. 8C shows a fluorescent image of nanoparticles labeled with quantum dots within a cell within a cell.

FIG. 8D is an overlay of FIGS. 8A-8C.

FIG. 9 shows a graph of gene transfection for various stimuli-responsive materials in some embodiments.

FIG. 10 is a graph of the fluorescence loss in photobleaching in certain examples of the present invention, at various pH levels.

FIG. 11A shows a transmission electron microscopy image of 30/70 (mol/mol) DMAEMA/HEMA nanoparticles crosslinked with 3 mol % TEGDMA.

FIG. 11B shows the results of cell viability assays of a control, and in 10/90, 20/80, and 30/70 (mol/mol) DMAEMA/HEMA nanoparticles crosslinked with 3 mol % TEGDMA.

FIG. 11C shows agarose gel electrophoresis for the assessment of plasmid DNA integrity extracted from 30/70 (mol/mol) DMAEMA/HEMA nanoparticles crosslinked with 3 mol % TEGDMA.

FIGS. 11D-F show the volume swelling ratios of (D) 10/90 (black), 20/80 (white), and 30/70 (diagonal) (mol/mol) DMAEMA/HEMA nanoparticles crosslinked with 3 mol % TEGDMA at pH 5.5; (E) DMAEMA/HEMA (30/70, mol/mol) nanoparticles crosslinked with 3 (black), 6 (white), and 9 (diagonal) mol % TEGDMA at pH 5.5; and (F) DMAEMA/HEMA (30/70, mol/mol) nanoparticles crosslinked with 3 mol % TEGDMA at pH 5.5 (black), 6.0 (white), 6.5 (diagonal), 7.0 (cross-hatched), and 7.4 (wave).

FIG. 12 shows a comparison of the (A) zeta potential and (B) elastic moduli of HEMA (100 mol %, black) and DMAEMA/HEMA hydrogels (10/90 (white), 20/80 (diagonal), and 30/70 (cross-hatched), mol/mol) were prepared with 3, 6, or 9 mol % TEGDMA.

FIG. 13 shows quantum dot-conjugated DMAEMA/HEMA (30/70, mol/mol) nanoparticles crosslinked with 3 mol % and 9 mol % TEGDMA which were endocytosed by HeLa cells as a function of time.

FIG. 14A shows HeLa cells which were treated separately with 10/90 (▪), 20/80 (▴), and 30/70 (•) (mol/mol) DMAEMA/HEMA nanoparticles crosslinked with 3 mol % TEGDMA encapsulating pDNA. Transfection with naked DNA (□), PLL/pDNA (Δ), and PEI/pDNA (∘) complexes were used as controls.

FIG. 14B shows transfection of HeLa cells treated separately with DMAEMA/HEMA (30/70, mol/mol) nanoparticles crosslinked with 3 (•), 6 (▴), and 9 (▪) mol % TEGDMA encapsulating pDNA and with HEMA nanoparticles crosslinked with 3 (∘), 6 (Δ), and 9 (□) mol % TEGDMA encapsulating pDNA.

FIGS. 14C-H show transfection of HeLa cells treated with DNA encapsulating, quantum dot-conjugated 30/70 (mol/mol) DMAEMA/HEMA nanoparticles crosslinked with 3 mol % TEGDMA after (C-E) 4 h and (F-H) 24 h of transfection.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention generally relates to compositions and methods responsive to environmental conditions. In some embodiments, the composition and methods relate to the delivery of a therapeutic or diagnostic agent. Compositions of the invention may be in the form of nanostructures, and this is an option in connection with any of the materials and compositions described below, even where not specifically noted.

Some embodiments of the invention involve materials that are responsive to changes in the surrounding environment, including changes in ion concentration. For example, one or more properties of the material may be affected by a change in pH and/or a change in the concentration of an ion in an environment to which the material is exposed. Such properties may include, for example, a dimension of the material (e.g., volume), the permeability of the material, and the like. In some embodiments, the invention also provides methods, including methods for delivering a therapeutic or diagnostic agent, with enhanced selectivity and sensitivity. For example, in cases where the material comprises a therapeutic or diagnostic agent, a change in the surrounding environment may cause the material to undergo a change which causes the rate of release of the agent from the material to increase or decrease.

An advantageous feature of the invention relates to the ability to provide materials and methods that may be responsive within physiological conditions. For example, materials described herein may be responsive within the blood pH range (e.g., between about 7.8 and about 7.0). Materials and methods of the invention may also exhibit enhanced sensitivity to small changes in the surrounding environment. For example, a material comprising a therapeutic or diagnostic agent may undergo a change in the release rate of the agent upon a pH change of the surrounding environment of less than 1.0 pH unit. Such properties may be advantageous, for example, when localized delivery of an agent is desired. The enhanced selectivity and/or sensitivity of materials and methods described herein may also allow for systemic administration of the materials to a subject (e.g., mammal), as described more fully below.

In some embodiments, materials of the invention may be administered to a mammal, wherein the material may be responsive to small changes (e.g., ion concentration, pH level) associated with one or more physiological conditions, i.e., the material may be “feedback-regulated,” rather than responsive to application of an external stimulus. For example, the material may be responsive (e.g., may swell) to a small change in ion concentration or pH associated with a tumor, myocardial infarction, and the like. The response of the material (e.g., swelling) may cause an agent associated with the material to be released from the material. It should be understood, however, that materials of the invention may also be used in combination with one or more external stimuli.

A material which is responsive to at least one change in a surrounding environment is a stimuli-responsive or ion-responsive material. A stimuli-responsive or ion-responsive material may be any material that exhibits at least one physical change (e.g., change in volume, release rate, color, etc.) in response to a stimulus. The stimulus may refer to an environmental characteristic such as ion concentration, pH, temperature, light, ionic strength, electric field, magnetic field, solvent composition, etc. In some cases, more than one stimulus may be used. In connection with materials of the invention responsive to a change in pH of the surrounding environment, the term “pH” is given its orginary meaning in the art and refers to the acidity or alkalinity of a solution. For example, pH may be general expressed as the effective concentration of hydrogen ions in solution. However, the pH may also be related to the concentration of other ions (e.g., O₂, CO₂, etc) present that may affect the acidity or alkalinity of the solution. In an illustrative embodiment, the microenvironment surrounding a tumor cell may exhibit a relatively lower pH value when compared to normal, non-tumor cells. In another embodiment, the presence of an excess amount of, for example, morphine in the body may affect the relative amounts of oxygen and carbon dioxide in the bloodstream, producing a change in pH.

As noted, in some embodiments, the present invention provides compositions for delivery of a therapeutic and/or diagnostic agent. In some embodiments, the composition may be a stimuli-responsive material (e.g., an ion-responsive material), which comprises a polymeric material and a therapeutic and/or diagnostic agent. In certain embodiments, the stimuli-responsive material comprises nanostructures (e.g., nanoparticles), wherein the therapeutic or diagnostic agent may be substantially contained (e.g., encapsulated) within the nanostructures such that, upon exposure to a stimuli, the agent may be released. For example, a composition comprising a plurality of nanoparticles may be exposed to an environment having a pH level that triggers release of the agent from the composition (e.g., by swelling of the nanoparticles). In certain cases, the agent may be delivered to a subject (e.g., mammal). It should be understood, however, that other methods and applications using stimuli-responsive materials as described herein are possible, such as diagnostic tools in a lab or related setting. Furthermore, although much of the discussion below describes the use of pH-responsive materials (e.g., pH-responsive nanostructures), other types of stimuli-responsive materials (e.g., ion-responsive) may be used in the methods as described herein.

In some embodiments, the stimuli-responsive materials may be used for delivering a therapeutic and/or diagnostic agent. In some embodiments, the agent may be delivered by systemic delivery to a subject. As used herein, “systemic delivery” generally refers to delivery of agents to the body as a whole. In other embodiments, the agent may be delivered by localized delivery to a subject. As used herein, “localized delivery” generally refers to delivery of agents to a particular area of the body, i.e., on a more targeted basis. Any suitable method and/or technique may be employed for delivering agents to body tissue, systemically or locally, as would be known to those of ordinary skill in the art. Existing techniques include, for example, oral administration, direct injection into body tissue, topical or transcutaneous administration, intravenous administration, etc.

Many current systematic delivery methods have a number of disadvantages. For example, when drugs are systemically administered at high levels, healthy tissue may be harmed, which in some cases, may cause serious side effects. In addition, systematic delivery of an agent may require a higher dosage of the agent, as compared to localized delivery, to achieve a desired level of the agent at an intended site and in some cases, localized delivery may not be possible (e.g., tumor in a location not accessible for localized delivery).

There are certain advantages of the present invention, in some embodiments, when employed in systematic delivery. For example, materials of the invention may be administered systemically but may be used to deliver a therapeutic or a diagnostic agent locally, i.e., delivery of the agent occurs primarily at a localized site rather than the body as a whole. In some cases, an agent that is associated with a material of the present invention may be released from the material only upon exposure to a stimulus. For example, the agent may be encapsulated in a pH-responsive nanostructure wherein the nanostructure has been administered to the body using systemic delivery (e.g., in the bloodstream). In some cases, the agent encapsulated in the pH-responsive material may be substantially contained within the material until the material is exposed to an environment having a particular pH value (e.g., a tumor site, which has a pH value which differs from the pH of the bloodstream), thereby effectively delivering the agent to a specific location within the subject. Some embodiments of the subject invention relate to pH-responsive materials that may be useful in the medical treatment of a disease. In some cases, the pH-responsive material may comprise nanoparticles and may encapsulate an agent. Methods for encapsulating an agent in a nanoparticle, as well as releasing the agent from the nanoparticle, are also provided. This is also an advantageous feature when administering agent which may be expensive as the required amount of agent (e.g., dosage) to be administered may be reduce, thereby reducing the cost of the treatment.

In some embodiments, where the invention is used in connection with delivery of a therapeutic or diagnostic agent to a subject or other environment, the method may comprise exposing a pH-responsive material to a first set of conditions (e.g., blood stream), wherein the pH-responsive material releases the agent at a first release rate. The pH-responsive material may then be exposed to a second set of conditions (e.g., tumor site), wherein the pH-responsive material releases the agent at a second release rate. The first release rate may be different from the second release rate. In some cases, the second release rate is greater than the first release rate.

In an illustrative embodiment, the pH-responsive material may be exposed to the bloodstream and may have a first release rate. The pH-responsive material, for example, may travel through the body and may encounter an environment comprising tumor cells, wherein the microenvironment surrounding the tumor cells comprises a relatively low pH as compared to the bloodstream. Exposure of the pH-responsive material to the tumor microenvironment (e.g., reduced pH level) may cause a change (e.g., an increase) in the release rate of the agent from the material (e.g., by swelling of the material). That is, the material may selectively deliver the therapeutic and/or diagnostic agent to locations comprising tumor cells, and may not significantly deliver the agent to locations lacking the tumor cells.

The “percent release” of an agent is generally described as a percentage of the agent released for a material versus the total amount of agent encapsulated in the material. In some cases, the percent release of an agent from a material may increase with time. In some instances, the percent release of the agent from a material may increase with a decrease in the pH level. In other cases, the percent release of an agent from a material may increase with an increase in the pH level. These factors may also affect the release rate of the agent from the material. As used herein, the term “release rate” refers to the ratio of the percent release of the agent from a material vs. time, that is, the percentage of agent associated with the material that is released from the material as a function of time. In some cases, the first release rate (e.g., prior to exposure of the material to a stimulus) is about 0% per hour, less than about 0.5% per hour, less than about 1% per hour, less than about 2% per hour, less than about 3% per hour, less than about 4% per hour, less than about 5% per hour, less than about 10% per hour, etc. The second release rate (e.g., following exposure of the material to a stimulus) may be greater than the first release rate by at least about 5%, at least about 10%, at least about 15%, at least about 20%, at least about 40%, at least about 50%, at least about 75%, at least about 100%, at least about 200%, or, in some cases, at least about 500%.

The percent release of an agent from a material may be about at least about 10%, at least about 20%, at least about 30%, at least about 40%, at least about 50%, at least about 60%, at least about 70%, at least about 70%, at least about 90%, at least about 99%, after a given period of time (e.g., about 1 hour, about 4 hours, about 8 hours, about 24 hours, etc.). In some cases, the release rate of an agent from a material may be at least about 70%, at least about 50%, at least about 40%, at least about 25%, at least about 20%, at least about 15%, at least about 10%, etc., over a 24 hour period. In other cases, the release rate of an agent from a material may be about at least 55%, at least about 50%, at least about 35%, at least about 25%, at least about 20%, at least about 15% at least about 10%, at least about 5%, etc. over a 4 hour period.

In some cases, the release rate of the stimuli-responsive materials (e.g., pH-responsive nanoparticles) is not affected by a change in temperature. That is, a change in temperature of about 1° C., of about 5° C., of about 10° C., of about 15° C., of about 25° C., etc., affects the release rate of the material by less than about 0.5%, less than about 1%, less than about 2%, less than about 5%, less than about 10%, etc. In other cases, the release rate of the stimuli-responsive nanostructure may be affected by a change in temperature.

As described herein, an agent that is encapsulated within a material may be released from the material upon exposure of the material to a stimulus, for example, a certain pH level. Various factors may affect the amount and/or rate at which the agent is released from the pH-responsive materials. For example, the selection and/or composition of the pH-responsive material, as well as the agent, may affect the percent release and/or the release rate of the agent from the material. For example, a strong interaction between the agent and the pH-responsive material may inhibit the release of the agent from the pH-responsive material as compared to instances where there is a weak interaction between the agent an the pH-responsive material.

The percent release and/or release rate of an agent from a pH-responsive material may vary depending on the properties of the pH-responsive material. For example, the swelling ratio of the material may affect the percent release and/or the release rate of the agent. Specifically, a larger swelling ratio may correspond to a higher percent release of an active agent as opposed to the percent release from a material with a smaller swelling ratio. The term, “swelling ratio,” as used herein, refers to the ratio of the average nanoparticle diameter under a second set of conditions to the average nanoparticle diameter under a first set of conditions. For example, for pH-responsive nanoparticles, the swelling ratio is the ratio of the nanoparticle diameter at a second pH to the nanoparticle diameter at a first pH (e.g., physiological pH, about pH 7.4). The term “swelling rate,” as used herein, refers to the rate in which a nanoparticle swells, and is generally given in nm/hr.

In some cases, the swelling of a material (e.g., nanostructure) comprises a volumetric change of at least about 0.1%, at least about 0.5%, at least about 1%, at least about 2%, at least about 5%, at least about 10%, at least about 20%, at least about 30%, at least about 50%, or more. That is, the volume of a material following swelling is greater than the volume of the material prior to swelling.

In some cases, the pH level may affect the percent release and/or the release rate of an agent from a pH-responsive material. For example, physical changes (e.g., swelling ratio) of a pH-responsive material, may depend on the pH level. For example, a small decrease in the pH level may lead to a small increase in the swelling ratio of a pH-responsive material as opposed to a larger decrease in the pH level which may lead to a larger increase in the swelling ratio. Therefore, the small decrease in the pH level may lead to less release of agent as opposed to the larger decrease in the pH level.

Because the percent release and/or release rate of an agent may be affected by the pH level, in certain embodiments, it may be useful to have a pH-sensitive material that has a low percent release and/or release rate a first pH level and a higher percent release and/or release rate at a second pH level. In some instances, the first pH level may be higher than the second pH level. In other instances, the first pH level may be lower that the second pH level. For example, the material may have a low percent release and/or release rate a physiological pH (e.g., about pH 7.4) and a higher percent release and/or release rate at a second pH level. These materials may be useful for systematic delivery of agents to a specific region of a body which may have a pH level different from the physiological pH level (e.g., a lower pH level near a tumor).

In some embodiments, the first and second conditions may comprise different pH values. The first pH value may be about 7.4. In some instances, the first pH value may differ from the second pH value by less than about 2 pH units, less than about 1 pH unit, less than about 0.75 pH units, less than about 0.5 pH units, less than about 0.4 pH units, less than about 0.3 pH units, less than about 0.2 pH units, less than about 0.1 pH units, or less than about 0.05 pH units. In other instances, the first pH value may be greater than or less than the second pH value. For example, the first pH value may be greater than or less than the second pH value by less than about 2 pH units, less than about 1 pH unit, less than about 0.75 pH units, less than about 0.5 pH units, less than about 0.4 pH units, less than about 0.3 pH units, less than about 0.2 pH units, less than about 0.1 pH units, or less than about 0.05 pH units.

In some cases, the pH-responsive materials may be selected and/or synthesized such that the agent may be released with a change in the pH level from about 7.5 to about 6.5, from about 7.4 to about 6.8, from about 7.4 to about 7.0, and from about 7.4 to about 7.2. The amount of agent that is released, i.e., the percent release of the agent, may be dependant on various factors including, but not-limited to, the pH-responsive material, time, pH, and the like.

As a specific example, a pH-sensitive material at pH level 7.4 may release about 16% of an encapsulated agent over a six hour period. However, if the pH level is changed to 6.8 after two hours, a total of about 45% of the encapsulated agent may be released over the next four hours (total time=six hours).

In some embodiments, the methods of the present invention are employed for delivery of a specific therapeutic and/or diagnostic agent. For example, the agent encapsulated in the stimuli-responsive material may be an antagonist to a therapeutic agent currently in subject's body. For example, an overdose of morphine in a subject may be associated with a drop in the subject's body pH-level. The agent encapsulated in the stimuli-responsive material may be such that it is an antagonist for the morphine currently in the subject's body. Therefore, delivery of the pH-responsive material to the body may allow for the release of the antagonist (e.g., due to the subject's decreased body pH) and the antagonist can sequester morphine in the body.

In another example, the agent encapsulated in the stimuli-response material may be a therapeutic agent (e.g., a chemotherapy agent) for delivery to a tumor. A change in the pH-level around the tumor may allow for the release of the agent encapsulated in the pH-responsive material at the site of the tumor.

The agent encapsulated in the stimuli-responsive material may also be a therapeutic agent (e.g., tissue plasminogen activator) for delivery to a site of a myocardial infarction. A change in the pH-level of the site of a myocardial infarction may allow for the release of an agent encapsulated in the pH-responsive material at the site of the myocardial infarction.

In yet another example, the agent encapsulated in the stimuli-responsive material may be a therapeutic agent (e.g., insulin) to help counter diabetic ketoacidosis. For example, a change in the pH-level of the body due to diabetic ketoacidosis may allow for the release of the agent from the pH-responsive material to the body.

The stimuli-responsive materials of the present invention may also be used for gene therapy. Gene therapy is the insertion of genes into cells and tissues to treat diseases in which a defective mutant allele is replaced with a functional one. Therefore, genes may be encapsulated in the stimuli-responsive material of the current invention to delivery the gene to a cell or tissue. The release of the gene may be correlated with exposure of the stimuli-responsive material to a stimulus.

The stimuli-responsive materials of the present invention may also be used to deliver an agent to a non-living subject. For example, the nanoparticles may be useful for releasing an agent into a water system (e.g., a pool) when a change in the pH level occurs.

The methods discussed above employ a composition for the delivery of therapeutic and/or diagnostic agents. The composition may comprise a pH-responsive material and a therapeutic or diagnostic agent, wherein the composition is comprised of pH-responsive nanostructures. The pH-responsive material may comprise hydrophilic repeating units and pH-responsive units. In some cases, the ratio of the hydrophilic to pH-responsive units is at least about 1.0:1.0. Examples of hydrophilic and pH-responsive units, the ratio of the units, and the properties of the materials as described herein.

In other embodiments, a composition may comprise a plurality of pH-responsive nanostructures which each comprise a base material (e.g., a polymeric material as discussed herein) and a therapeutic and/or diagnostic agent associated (e.g., encapsulated) in the base material. The composition may have a first release rate of the agent from the material under a first set of conditions and a second release rate of the agent from the material under a second set of conditions. The conditions may be physiologically-compatible.

It should be understood, however, that while the discussion herein primarily focuses on compositions comprised of pH-responsive materials and/or nanoparticles, this is by way of example only, and other stimuli-responsive materials and/or nanostructures may be used in place of or in conjunction with the pH-responsive materials and/or nanoparticles, in certain embodiments. In particular, ion-responsive materials may be used in place of pH-responsive materials.

In some embodiments of the present invention, the stimuli-responsive material comprises a polymeric material. A “polymer,” as used herein, is given its ordinary meaning as used in the art, i.e., a molecular structure comprising one or more repeat units (e.g., monomers), connected by covalent bonds. The polymeric material may comprise one or more repeating units, and in some cases, a crosslinking agent. The polymeric material may be synthesized using any number of techniques, including use of an initiator. Repeating units, synthetic methods, crosslinking agent, and initiators are discussed more herein.

The stimuli-responsive material may comprise a polymeric material which comprises of one or more repeating units such as copolymers, terpolymers, higher order copolymers, and the like. The polymeric materials may be in linear, branched, or grafted form, the grafted chains being exclusively one polymer or copolymers of the above, ionically bound, or complexed by hydrogen bonds.

A “repeating unit” is a unit within the polymeric material that occurs more than once. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks. The repeating units of a certain type may or may not be situated relative to each other (e.g., the ordering of the repeating units may or may not be random).

In some cases, one or more of the repeating units of the polymer may be pH-responsive repeating units and one or more of the repeating units may be non-pH-responsive repeating units. A “pH-responsive repeating unit” is a repeating unit which responds to a change in pH, thereby forming a pH-responsive polymer. A “pH-responsive polymer” is a polymer that responds to a change in pH. In some cases, a pH-responsive repeating unit is co-polymerized with a non-pH-responsive repeating unit. The co-repeating units may be selected accordingly to affect the solubility, sensitivity, physical characteristics, and the like, of the stimuli-responsive material. Other types of stimuli-responsive repeating units may be employed as in the stimuli-responsive polymers such that the polymers respond to a stimulus other than pH (e.g., light, temperature).

Examples of pH-responsive repeating units that may be used to form pH-responsive polymeric materials include, but are not limited to, acrylic acid, 2-(dimethylamino)ethyl methacrylate (DMAEMA), methacrylic acid, maleic anhydride, maleic acid, 2-acrylamido-2-methyl-1-propanesulfonic acid, N-vinyl formamide, N-vinyl acetamide, aminoethyl methacrylate, phosphoryl ethyl acrylate, or phosphoryl ethyl methacrylate. pH-Responsive polymeric material may also comprise polypeptides from amino acids, polysaccharides, nucleic acids, etc. pH-Responsive repeating units generally comprise pendant pH-responsive groups such as —OPO(OH)₂, —COOH, and/or —NH₂ groups. In a specific example, the pH-responsive repeating unit is DMAEMA.

The non-pH-responsive polymeric materials and/or repeating units may be chosen such that they do not respond to an external stimulus. In some cases, the non-pH-responsive repeating units and/or polymeric materials may be hydrophilic. A hydrophilic polymeric material is generally a polymeric material that attracts water while a hydrophobic polymer is a polymeric material that generally repels water. Examples of hydrophilic polymers include, but are not limited to poly(vinyl alcohol), poly(vinyl pyrrolidone), gelatin, cellulose ethers, poly(oxazolines), poly(vinylacetamides), partially hydrolyzed poly(vinyl acetate/vinyl alcohol), poly(acrylic acid), poly(acrylamide), poly(alkylene oxide), sulfonated or phosphated polyesters and polystyrenes, casein, zein, albumin, chitin, chitosan, dextran, pectin, collagen derivatives, collodian, agar-agar, arrowroot, guar, carrageenan, tragacanth, xanthan, rhamsan and the like. A specific example of a hydrophilic non-pH-responsive polymeric material and/or repeating unit is hydroxyethyl methacrylate (HEMA). The terms, “hydrophobic” and “hydrophilic” are given their ordinary meaning in the art and, as will be understood by those skilled in the art, in many instances herein, these are relative terms. Although specific parameters or limitations on the meaning of a “hydrophobic material” (e.g., repeating unit) would be inappropriate given different relative hydrophobicities required for different release profiles, in general, a hydrophobic polymeric unit is one that, when formed into a material suitable for a contact angle measurement, will result in a water contact angle of greater than about 50°.

The polymerization of the repeating units may take place under any suitable reaction condition as known to those of ordinary skill in the art. The conditions that may be varied include, but are not limited to, temperature, solvent, concentration of the reactants, and the like. In some instances, the components of the polymerization reaction (e.g., one or more repeating unit, crosslinking agent, and/or initiator) may be mixed together in a solution. The solution may comprise of one or more solvents such as water, toluene, hexane, and the like. As discussed below, in some cases, the conditions of the polymerization may be chosen such that the polymeric material forms nanostructures. For example, in some cases, a crosslinking agent may be provided which aids in the formation of nanostructures, as described herein.

In some cases, the polymerization reaction comprises reacting one or more repeating units and an initiator. An “initiator” is any reagent and/or condition that cause the polymerization of the repeating units to begin. In some cases, the initiator may be a photoinitiator, and the polymerization of the repeating units in the presence of the initiator may begin upon exposure of the mixture to a certain wavelength of light. The initiator may be combined with the repeating units in any ratio that is suitable. For example, the ratio of initiator to repeating units may be such that the polymeric material formed is of a certain length. Non-limiting examples of initiators include 2,2-dimethoxy-2-phenylacetophenone (DMPAP) and N,N′-azobisisobutyronitrile (AIBN). In some instances, the initiator may be provided in at least about 0.1 mol %, at least about 0.5 mol %, at least about 1 mol %, at least about 2 mol %, at least about 5 mol %, at least about 10 mol %, or more, of the total repeating unit amount.

In certain cases, the polymerization reaction may further comprise a crosslinking agent. A “crosslinking agent” is any compound that allows for the crosslinking of one or more polymer chains. The presence of a crosslinking agent during the polymerization may aid in the formation of particles (e.g., nanoparticles) of the polymeric material. The crosslinking agent employed may be any suitable reagent that is compatible with the repeating units and/or the initiator, for example a polyunsaturated co-repeating unit. In some instances, the crosslinking agent is not compatible with the initiator and/or the repeating units. Non-limiting examples of crosslinking agents include tetraethylene glycol dimethacrylate (TEGDMA), N,N′-methylenebisacrylamide (MBA), and the like. The crosslinking agent may be combined with the repeating units in any ratio that allows for the formation of a nanomaterial. In some instances, the crosslinking agent is provided in at least about 0.1 mol %, at least about 1 mol %, at least about 2 mol %, at least about 3 mol %, at least about 5 mol %, at least about 10 mol %, at least about 20 mol %, or more, of the total repeating unit amount.

The ratio of the initiator and/or the crosslinking agent to the repeating units may be altered to affect the physical characteristics of the stimuli-responsive nanoparticles. A change in the ratio(s) may affect the elasticity, volume swelling ratio, swelling rate, stability, size, and the like, of the nanoparticles.

In a particular embodiment, a pH-responsive material comprises pH-responsive repeating units, 2-(dimethylamino)ethyl methacrylate (DMAEMA) and non-pH responsive repeating units, hydroxyethyl methacrylate (HEMA). The ratio of DMAEMA to HEMA may be varied and affect the properties of the nanoparticles. The ratio of HEMA:DMAEMA may be at least about 1.0:1.0, at least about 2.0:1.0, at least about 5.0:1.0, at least about 10.0:1.0, at least about 20.0:1.0, or more. In some embodiments, the material comprising DMAEMA and HEMA may be crosslinked using TEGDMA. TEGDMA may be provided in at least about 1 mol %, at least about 3 mol %, at least about 5 mol %, at least about 10 mol %, at least about 20 mol %, or more, of the total amount of DMAEMA and HEMA. The presence of TEGMDA may aid in the formation of nanoparticles. As described herein, the average diameter of the nanoparticles may be about 100 nm, about 150 nm, about 200 nm, about 230 nm, about 250 nm, about 300 nm, about 350 nm, about 500 nm, or greater.

In some embodiments, increasing the ratio of pH-responsive units to non-pH-responsive repeating unit may increase or decrease the swelling rate, swelling ratio, release rate, release ratio, elasticity, etc., of the material. For example, increasing the ratio of DMAEMA:HEMA may increase the sensitivity of the polymeric material to changes in pH. As a specific example, the ratio of DMAEMA:HEMA may be increased (e.g., from 5:95 to 10:90) to increase the swelling ratio of the nanoparticles (e.g., from 1.4% to 1.7%, respectively). In some cases, the swelling rate may also increase with increasing DMAEMA content. As a specific example, the ratio of DMAEMA:HEMA may be increased (e.g., from 5:95 to 10:90) to increase the swelling rate of the nanoparticles at a certain pH (e.g., from 25 nm/hr to 40 nm/hr at pH 6.8).

In some embodiments, increasing the amount of crosslinking agent may increase or decrease the swelling ratio, swelling rate, release rate, release ratio, elasticity, etc., of the material. For example, an increase in the TEGDMA ratio may reduce the elasticity of the nanoparticles and/or increase the stability of the nanoparticles. A change in the TEGDMA ratio may also decrease the elasticity of the nanoparticles and/or decrease the stability of the nanoparticles.

In some cases, varying the ratio of components of a material may increase of decrease the elasticity of the material. In some cases, increasing the amount of crosslinking agent ratio may increase or decrease the elasticity of the material. For example, an increase in the amount of TEGDMA provided may increase the elastic moduli of the material. The term, “elastic modulus,” is given its ordinary meaning in the art and refers to a mathematical description of an object or material's tendency to be deformed elastically (i.e., non-permanently) when a force is applied to it. In some instances, the elastic modulus may be reported as Young's modulus (E) which it is defined as the ratio of tensile stress to tensile strain. The elastic moduli of a material may be determined using methods known to those of ordinary skill in the art, for example, by placing a material under increasing stresses, and measuring the resulting strains.

As noted, in some embodiments, a stimuli-responsive material of the invention comprises a nanostructure. A “nanostructure” refers to any material of intermediate size between molecular and microscopic (e.g., micrometer-sized) structures, for example, a nanoparticle. In some embodiments, a nanostructure may respond to a stimulus by either increasing in size (e.g., swelling) or decreasing in size (e.g., contracting). In some instances, the stimulus may be a change in pH. For example, the stimuli-responsive material may comprise nanoparticles that swell or contract when exposed to a change in pH.

Without wishing to be bound by theory, the size of a particle may alter the delivery (e.g., loss of payload, drug efflux, aggregations, delivery to desired location, etc.) of an agent from the particles. In some cases, larger particles may lose their payload more quickly than smaller particles and/or a drug efflux may be more rapid from smaller particles than larger particles. Smaller particles, in some cases, may be more likely to aggregate than larger particles. The size of the particle may affect the distribution of the particles throughout the body. For example, larger particles injected into a bloodstream may be more likely to be lodged in small vessels than smaller particles. In some instances, larger particles may be less likely to cross biological barriers (e.g., capillary walls) than smaller particles. The size of the particles used in a delivery system should be selected based on the application, and will be readily known to those of ordinary skill in the art. For example, particles of smaller size (e.g., <200 nm) may be selected if systematic delivery of the particles throughout a patient's bloodstream is desired. As another example, particles of larger size (e.g., >200 nm) may be selected if sequestering of the particles by a patient's reticuloendothelial system upon injection is desired (e.g., sequestering of the particles in the liver, spleen, etc.). The desired length of time of delivery may also be considered when selecting particle size. For example, smaller particles may circulate in the blood stream for longer periods of time than larger particles.

In some cases, a particle may be a nanoparticle, i.e., the particle has a characteristic dimension of less than about 1 micrometer, where the characteristic dimension of a particle is the diameter of a perfect sphere having the same volume as the particle. The plurality of particles, in some embodiments, may be characterized by an average diameter (e.g., the average diameter for the plurality of particles). In some embodiments, the diameter of the particles may have a Gaussian-type distribution. In some cases, the plurality of particles may have an average diameter of less than about 500 nm, less than about 400 nm, less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles may have an average diameter of about 5 nm, about 10 nm, about 30 nm, about 50 nm, about 100 nm, about 150 nm, about 200 nm, about 250 nm, about 300 nm, about 400 nm, about 500 nm, or greater. In some cases, the nanoparticles have an average size between about 1 nm and about 1 um, between about 10 nm and about 500 nm, between about 50 nm and about 400 nm, or between about 100 nm and about 300 nm. In a particular embodiment, the nanoparticles have an average nanoparticle size between about 100 nm and about 250 nm.

It should be understood that the average nanoparticle size of a nanoparticle composition may be determined by measuring an average cross-sectional dimension (e.g., diameter for substantially spherical nanoparticles) of a representative number of nanoparticles. For example, the average cross-sectional dimension of a substantially spherical nanoparticle is its diameter; and, the average cross-sectional dimension of a non-spherical nanoparticle is the average of its three cross-sectional dimensions (e.g., length, width, thickness), as described further below. The nanoparticle size may be determined using microscopy techniques, such as scanning electron microscope or transmission electron microscopy techniques or optical techniques, such as dynamic light scattering.

In some embodiments, an agent may be encapsulated in a nanoparticle during formation of the nanoparticle. Encapsulated refers to the agent being substantially contained within an interior of a nanoparticle. For example, an agent is substantially contained (e.g., encapsulated) by the nanoparticle wherein the agent does not sufficiently dissociate from the nanoparticle until the nanoparticle is exposed to a stimulus (e.g., change in pH, ions), as described herein.

In some cases, the agent may be encapsulated into a polymeric material by adding an agent to a solution comprising repeating units, prior to the polymerization of the repeating units. For example, a first solution may be formed which comprises an agent and one or more repeating units. The first solution may be exposed to conditions which promote the polymerization of the repeating units and the agent may be encapsulating within the nanoparticles during formation of the nanoparticles. As a specific example, a first solution comprising an agent, a first repeating unit, and a second repeating unit may be provided as an aqueous phase. A non-aqueous phase comprising a crosslinking agent an initiator may be added to the aqueous phase. The resulting nanoparticles encapsulate the agent.

In other embodiments, an agent may also be encapsulated into nanoparticles after the nanoparticles have been formed. For example, the nanoparticles and agent may be mixed together in a solution. That is, the nanoparticles may be transferred from a solution where they are in a contracted state into a solution comprising the agent in which the nanoparticles will swell. The swelling of the nanoparticles in the presence of the agent may allow for the encapsulation of the agent. The nanoparticles may then be returned to a contracted state by transferring the nanoparticles into a solution that promotes contraction of the nanoparticles. This process may trap the agent within the nanoparticles until the nanoparticles are again place under conditions where the nanoparticles will swell. The encapsulation of agent may also be accomplished through changes in temperature, pH, ionic strength, and the like.

In some cases, the ratio of the agent to the repeating unit of the material may affect the percent loading of the therapeutic agent in the particle and/or the mean size of the particle. For example, an increase in the percent weight of the agent provided to the percent weight of the repeating units may increase the percent loading of the agent within the particles formed. However, in some cases, the percent loading of the agent in the particles formed may or may not be related to the weight percent of the agent provided (e.g., during formation of the particles). In some cases, the percent weight of the agent provided (e.g., in a solution) to the agent and the repeating units is at least about 5%, at least about 10%, at least about 20%, at least about 30%, at least about 40%, at least about 50%, at least about 60%, at least about 70%, at least about 80%, at least about 90%, at least about 100%, or greater, than the weight of the repeating units. The percent loading of the agent comprised in the plurality of particles may be greater than about 0.01%, greater than about 0.05%, greater than about 0.1%, greater than about 0.5%, greater than about 1%, greater than about 2%, greater than about 5%, greater than about 10%, greater than about 15%, greater than about 20%, greater than about 25%, greater than about 30%, greater than about 35%, greater than about 40%, greater than about 45%, greater than about 50%, greater than about 55%, or greater, the total amount of repeating units. In some cases, at least about 100%, at least about 90%, at least about 80%, at least about 70%, at least about 60%, at least about 50%, at least about 25%, at least about 10%, etc., of the agent provided is encapsulated in the nanoparticles.

The controlled formation of nanoparticles may be achieved using a number of techniques, for example, emulsion, suspension, interfacial polymerization, and the like, as will be known to those or ordinary skill in the art. In some cases, the reaction is carried out in an oil in water (O/W) emulsion. For example, the repeating units in solution may be added to a water phase that comprises the initiator, the crosslinking agent, and/or the therapeutic and/or diagnostic agent. The O/W emulsion may then be agitated using any suitable technique (e.g., sonication). Formation of the nanoparticles in an emulsion may aid in the formation of uniform size nanoparticles.

In some instances, the nanoparticles may be stabilized. This may be accomplished through various techniques, including addition of a stabilization agent. A non-limiting example of a stabilization agent is polyvinyl pyrrolidone.

In some cases, the pH-responsive material may also be referred to an ion-responsive material. For example, the pH-responsive material may be considered responsive due to a change in the ions around the pH-responsive material. In a specific example, the pH-responsive material that is in a blood system may be considered responsive to the concentration of ions within the blood stream (e.g., O₂, CO₂) and therefore, the material may be defined as an ion-reponsive material.

As used herein, “substantially,” in connection with an agent being contained within a material prior to exposure of the material to a stimulus, means that at least about 10%, at least about 25%, at least about 35%, at least about 50%, at least about 60%, at least about 75%, at least about 85%, or at least about 90% of the agent remains encapsulated in and/or associated within the particle before exposure to a stimulus. In some cases, less than about 50%, less than about 40%, less than about 30%, less than about 20%, less than about 10%, less than about 5%, or less than about 1%, of the agent is released from the material prior to exposure to a stimulus.

Those of ordinary skill in the art would, based on the teachings of the specification, be able to select materials suitable for use in a particular application. For example, a simple screening test for selecting such materials may involve the synthesis of polymers having various ratios of repeating units and/or a crosslinking agent and placing the resulting particles in an solution (e.g., aqueous solution) having a particular pH. Observation of the behavior of such particles within the aqueous solution of a certain pH may provide information regarding the swelling behavior, release rate, etc., of the particles at that pH. For example, the ratio of the one or more repeating units within the polymeric material may be varied in order to alter the physical properties of the polymeric material, such as size, agglomeration, and the like. In some instances, the ratio of the non-pH-responsive repeating units may be increased with respect to the pH-responsive repeating units. For example, the ratio of the non-pH-responsive repeating units (e.g., hydrophilic repeating units) to pH-responsive repeating units may be at least about 1.0:1.0, at least about 2.0:1.0, at least about 3.0:1.0, at least about 5.0:1.0, at least about 10.0:1.0, at least about 20.0:1.0, at least about 50.0:1.0, or more.

Many aspects of the invention involve delivery of an agent with a material (e.g., nanoparticles) to a site of delivery. “Site of delivery,” as used herein, refers to any location within a subject where the agent is desirably released. A site of delivery can include a specific type of tissue, specific organ, node, tumor, or the like, or can simply be the circulatory system itself if that is desired. In some cases, the site of delivery may comprise a stimulus (e.g., a change in pH) as compared to areas which are not the site of delivery (e.g., the bloodstream). Of course, where materials are delivered to a site of delivery in accordance with the invention, not all of the material administered need necessarily go to that site and in some embodiments, much of it does not go to the desired site of delivery. Of those in ordinarily skilled in the art would understand, a particle carrying an agent, desirably delivered to the bloodstream, a tumor, or another intended site of delivery, will include a substantial fraction, typically a majority, that will not be delivered to that site but will be captured by the spleen and/or liver of a subject. Nonetheless, some of the particles will go to the site of delivery.

In some cases, the agent may be substantially contained within the material until the material is at a site of delivery (e.g., such as a tumor, wherein there is a change in the environmental surrounding about the tumor, as described more herein). In some instances, less than about 90%, less than about 80%, less than about 70%, less than about 60%, less than about 50%, less than about 40%, less than about 30%, less than about 20%, less than about 15%, less than about 10%, less than about 5%, less than about 1%, or less, agent is released from the material prior to the material becoming localized at a particular targeting site. In some cases, however, the agent may be released without delivery to a specific site, for example, within the bloodstream.

Agents that may be encapsulated in the stimuli-responsive materials include therapeutic, diagnostic, and/or prophylactic agents. In some embodiments, the materials may comprise more than one agent, for example, at least two agents, at least three agents, at least for agents, or more. In some embodiments of the present invention, the agent is delivered to the target site where release occurs as function of change in permeability due to the interaction of a stimulus with the stimulus-responsive material. Agents can be proteins or peptides, polysaccharides, lipids, nucleic acid molecules, or synthetic organic molecules. Examples of hydrophilic molecules include most proteins and polysaccharides. Examples of hydrophobic compounds include some chemotherapeutic agents such as cyclosporine and taxol. Agents that can be delivered include hormones, chemotherapeutics; antibiotics, antivirals, antifungals, vasoactive compounds, immunomodulatory compounds, vaccines, local anesthetics, antiangiogenic agents, antibodies, neurotransmitters, psychoactive drugs, drugs affecting reproductive organs, and antisense oligonucleotides. Diagnostic agents include gas, radiolabels, magnetic particles, radioopaque compounds, and other materials known to those skilled in the art. A specific example of a therapeutic agent is paclitaxel.

Although agents described here are primarily with reference to therapeutic and diagnostic agents, it should be understood that the nanostructures can be used for delivery of a wide variety of agents. Non-limiting examples include cosmetic agents, fragrances, dyes, pigments, photoactive compounds, and chemical reagents, and other materials requiring a controlled delivery system. Other examples include metal particles, biological polymers, nanoparticles, biological organelles, and cell organelles.

In one set of embodiments, the stimuli-responsive material may be biocompatible, i.e., the material that does not typically induce an adverse response when inserted or injected into a living subject, for example, without significant inflammation and/or acute rejection of the polymer by the immune system, for instance, via a T-cell response. It will be recognized, of course, that “biocompatibility” is a relative term, and some degree of immune response is to be expected even for materials that are highly compatible with living tissue. However, as used herein, “biocompatibility” refers to the acute rejection of material by at least a portion of the immune system, i.e., a non-biocompatible material implanted into a subject provokes an immune response in the subject that is severe enough such that the rejection of the material by the immune system cannot be adequately controlled, and often is of a degree such that the material must be removed from the subject. One simple test to determine biocompatibility is to expose a material to cells in vitro; biocompatible materials are materials that typically will not result in significant cell death at moderate concentrations, e.g., at concentrations of about 50 micrograms/10⁶ cells. For instance, a biocompatible material may cause less than about 20% cell death when exposed to cells such as fibroblasts or epithelial cells, even if phagocytosed or otherwise uptaken by such cells.

In certain embodiments, the biocompatible material is biodegradable, i.e., the material is able to degrade, chemically and/or biologically, within a physiological environment, such as within the body. For instance, the material may be one that hydrolyzes upon exposure to water (e.g., within a subject), the materials may degrade upon exposure to heat (e.g., at temperatures of about 37° C.). Degradation of a material may occur at varying rates, depending on the material used. For example, the half-life of the material (the time at which 50% of the material is degraded into smaller moieties, for example, monomers and/or other non-polymeric moieties) may be on the order of days, weeks, months, or years, depending on the polymer. The materials may be biologically degraded, e.g., by enzymatic activity or cellular machinery, in some cases, for example, through exposure to a lysozyme (e.g., having relatively low pH). In some cases, the material (e.g., a polymer) may be broken down into monomers and/or other non-polymeric moieties that cells can either reuse or dispose of without significant toxic effect on the cells.

The invention further comprises preparations, formulations, kits, and the like, comprising any of the compositions as described herein. In some cases, treatment of a disease (e.g., a tumor) may involve the use of compositions or “agents” as described herein. That is, one aspect of the invention involves a series of compositions (e.g., pharmaceutical compositions) or agents useful for treatment of a disease (e.g., cancer or a tumor). These compositions may also be packaged in kits, optionally including instructions for use of the composition for the treatment of such conditions. These and other embodiments of the invention may also involve promotion of the treatment of a disease (e.g., cancer or tumor) according to any of the techniques and compositions and combinations of compositions described herein.

Examples of subjects include humans, animals (e.g., mammals), and the like. Non-limiting examples of animals include pigs, monkeys, goats, sheep, and dogs. Thus, as can be readily appreciated by one of ordinary skill in the art, the methods of the present invention are suited for use with any animal.

The following examples are included to demonstrate various embodiments of the invention. Those of ordinary skill in the art should, in light of the present disclosure, appreciate that many changes can be made in the specific embodiments which are disclosed and still obtain a like or similar result without departing from the spirit and scope of the invention. Accordingly, the following examples are intended to illustrate certain embodiments of the present invention, but do not exemplify the full scope of the invention.

### EXAMPLES

### Example 1

The following example gives information regarding the preparation of DMAEMA/HEMA nanoparticles. Pluronic F68 (BASF Corporation, Mount Olive, N.J., USA, 150 mg) was dissolved in 10 mL of distilled water for the water phase. Distilled water (18.2 MΩ) used in all experiments was from a Milli-Q purification system (Millipore Corp., Billerica, Ma., USA). To form an oil in water (O/W) emulsion, 100 uL of a DMAEMA (Acros, Morris Plains, N.J., USA) and HEMA (Acros, Morris Plains, N.J., USA) solution (mixed at different molar ratios of 5/95 and 10/90, respectively) was added to the water phase. Three different mol % TEGDMA (Fluka, St. Louis, Mo., USA) and 1 mol % DMPAP (Aldrich, St. Louis, Mo., USA) were added simultaneously to the water phase. The solution was sonicated (200 W, 20 kHz; Digital sonifier 250, Branson Ultrasonics Corp., Danbury, Conn., USA) in a laminar flow hood over an ice bath for 10 min. Following sonication, the DMAEMA/HEMA emulsion was exposed to a 365 nm UV energy source with 21,700 μW/cm² intensity (Model B-100 AP/R, UVP Inc., Upland, Calif., USA) for 90 seconds for photoinitiation. DMAEMA/HEMA nanoparticles were collected by high-speed centrifugation at 39000×g for 20 min (Sorvall RC26 Plus, SA-600 rotor; Thermo Fisher Scientific Inc., Waltham, Mass., USA). The nanoparticles were washed twice with pH 7.4 phosphate buffer to remove residual surfactant at the aforementioned operating conditions for the centrifuge. Monobasic and dibasic sodium phosphates used to prepare various pH phosphate buffers were purchased from Sigma (St. Louis, Mo., USA). For the preparation of paclitaxel-loaded nanoparticles, paclitaxel was directly added to the DMAEMA/HEMA solution at 1 mM final concentration. It was followed by sonication and centrifugation, as described above. The nanoparticles were freeze-dried (FTS systems, Inc., Stone Ridge, N.Y., USA) and stored at 4° C. for later use.

### Example 2

The following example describes the characterization of DMAEMA/HEMA nanoparticles. The size and morphology of DMAEMA/HEMA nanoparticles were analyzed by dynamic light scattering (ZetaPALS; Brookhaven Instrument, Holtsville, N.Y., USA) and transmission electron microscopy (JEOL 2100; JEOL Ltd., Tokyo, Japan). For TEM images, 0.05 wt % aqueous solution of DMAEMA/HEMA nanoparticles was dropped onto a copper grid (300 mesh, TED PELLA Inc., Redding, Calif., USA) supporting a thin film of amorphous carbon. The excess liquid was swept away with filter paper and the grid was dried in the hood. To obtain the effective surface charge, 0.1 mg/mL of DMAEMA/HEMA nanoparticles in an aqueous solution was measured by electrophoresis (ZetaPALS; Brookhaven Instrument, Holtsville, N.Y., USA) at different pHs in N-[tris(hydroxymethyl)methyl]-2-aminoethalesulfonic acid (TES) buffer (pH 6.8, 7.0, 7.2, and 7.4, respectively).

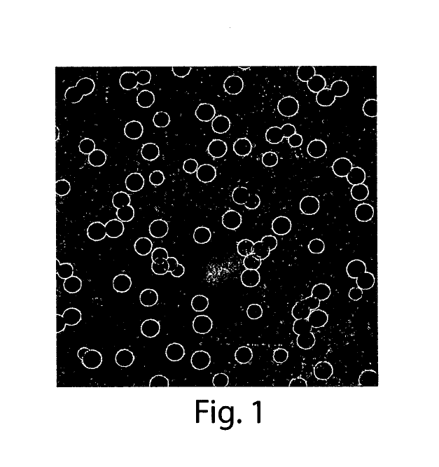

The nanoparticles were prepared with different molar ratios of DMAEMA to HEMA and varying TEGDMA concentrations. DMAEMA/HEMA nanoparticles were analyzed by TEM and dynamic light scattering. FIG. 1 depicts the TEM image of 10/90 DMAEMA/HEMA nanoparticles crosslinked by 3 mol % TEGDMA (scale bar=500 nm). Photomicroscopic examination of the nanoparticles reveals a uniform, smooth surface morphology. The average diameter of DMAEMA/HEMA nanoparticles was determined to be 230±30 nm by evaluating nanoparticles in at least five electron microscopic fields. The average diameter of nanoparticles with different molar ratios (either 5/95 or 10/90) and different TEGDMA concentrations (either 3, 5, or 10 mol %) were not significantly different after synthesis or when hydrated at pH 7.4. These results were confirmed by dynamic light scattering.

### Example 3

The following example describes the swelling studies completed on the DMAEMA/HEMA nanoparticles. Monodispersed DMAEMA/HEMA nanoparticles that exhibit pH sensitivity were successfully prepared by O/W emulsification and subsequent photopolymerization, as discussed above. Swelling studies of nanoparticles from different formulations were performed in phosphate buffer adjusted to the appropriate pH. The swelling studies of the DMAEMA/HEMA nanoparticles were performed in a buffered solution of known pH, composition, and temperature. The pHs of the buffered solutions were 6.8, 7.0, 7.2, and 7.4, respectively. A predetermined amount of freeze-dried DMAEMA/HEMA nanoparticles were put into a scintillation vial containing 10 mL of buffered medium. Samples were placed on a shaker (OS-500 orbital shaker, VWR, West Chester, Pa., USA) with a shaking rate of 100±1 rpm in an incubator maintained at 37° C. The average nanoparticle diameter was measured by dynamic light scattering at 0, 1, 2, 4, and 24 h.

The data is presented in the form of the volume swelling ratio, which may be described as the ratio of the nanoparticle diameter at a specific pH to the diameter at pH 7.4 (Q=d_{pH}/d_{pH 7.4}). The volume swelling ratio was measured as a function of time to analyze the effect of DMAEMA content, pH, and TEGDMA concentration on nanoparticle degradation, pH sensitivity, and matrix elasticity.

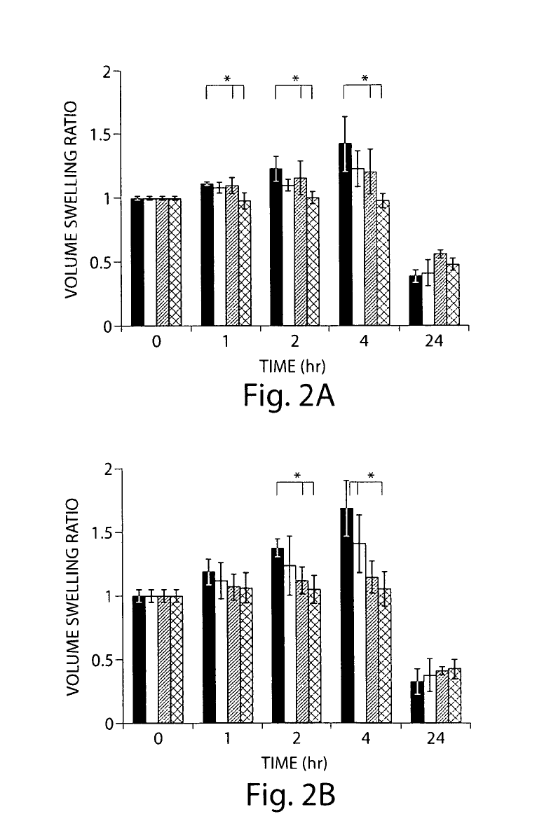

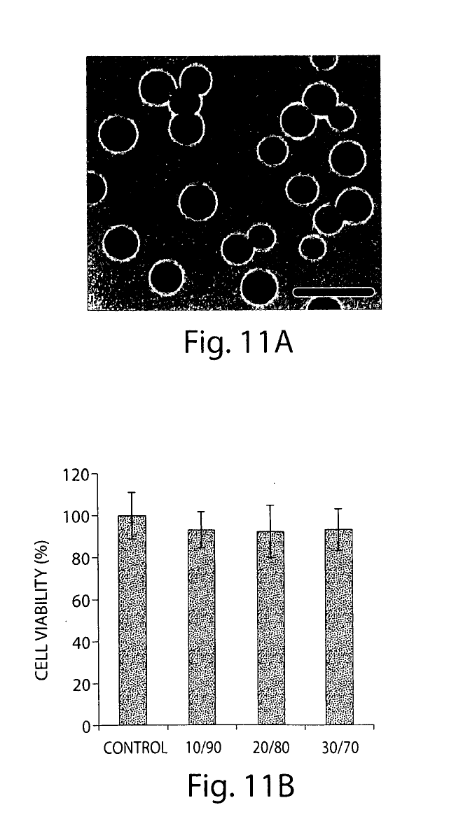

The swelling behavior of DMAEMA/HEMA nanoparticles varied by formulation and pH conditions. FIG. 2 shows the average nanoparticle diameters of DMAEMA/HEMA nanoparticles with molar ratios of 5/95 (FIG. 2a) and 10/90 (FIG. 2b) DMAEMA/HEMA after swelling as a function of pH and time. The volume swelling ratios are given according to the pH level of the solution. The pH levels studied were pH 6.8 (black), 7.0 (white), 7.2 (diagonal), and 7.4 (cross-hatched). The error bars shown correlated to the standard deviation of the mean, where n=3. The statistical significance was calculated relative to pH 7.4 at a specific time point, p<0.01. It was observed that the swelling ratio of nanoparticles significantly increased with decreasing pH values for both 5/95 and 10/90 DMAEMA/HEMA molar ratios. The diameter of the 10/90 DMAEMA/HEMA nanoparticles at pH 6.8 (394 nm) was larger than at pH 7.4 (246 nm) after 4 hours of swelling.

The nanoparticle with 5% DMAEMA (FIG. 2A) had lower volumetric swelling relative to the 10% DMAEMA nanoparticles (FIG. 2B). For example, 5/95 DMAEMA/HEMA nanoparticles swelled 1.4±0.2% after 4 hours at pH 6.8 while the 10/90 DMAEMA/HEMA nanoparticles (FIG. 2B) swelled 1.7±0.2% under the same conditions. Due to the increased concentration of protonated groups, the higher DMAEMA content nanoparticle may results in larger volumetric swelling.

The pH-sensitivity of each formulation may be addressed by looking at the swelling rate, or change in diameter as a function of time. The swelling rate after 4 hours for 5/95 DMAEMA/HEMA nanoparticles crosslinked with 3 mol % TEGDMA at pH 6.8, 7.0, 7.2, and 7.4 was 25, 14, 13, and ˜1 nm/hr, respectively. The swelling rate for these nanoparticles increased with DMAEMA content. Thus, the swelling rate after 4 hours for 10/90 DMAEMA/HEMA nanoparticles crosslinked with 3 mol % TEGDMA at pH 6.8, 7.0, 7.2, and 7.4 was 40, 25, 10, and 4.3 nm/hr, respectively. Hence, even a small pH change of 0.2 units results in an approximate ten-fold increase in swelling for 5/95 DMAEMA/HEMA nanoparticles or a two-fold increase in swelling for 10/90 DMAEMA/HEMA nanoparticles.

After 24 hours both 5/95 and 10/90 DMAEMA/HEMA nanoparticles degraded to approximately half their initial diameter. This may be due to bulk erosion, where the rate of diffusion is less than the rate of hydrolysis. The erosion number is the ratio of the time for water to diffuse into the gel to the time required for hydrolysis. For polyesters, the erosion number is typically less than 1 (a hydrolysis dominated regime). Therefore, changes in surface area may not significantly affect degradation.

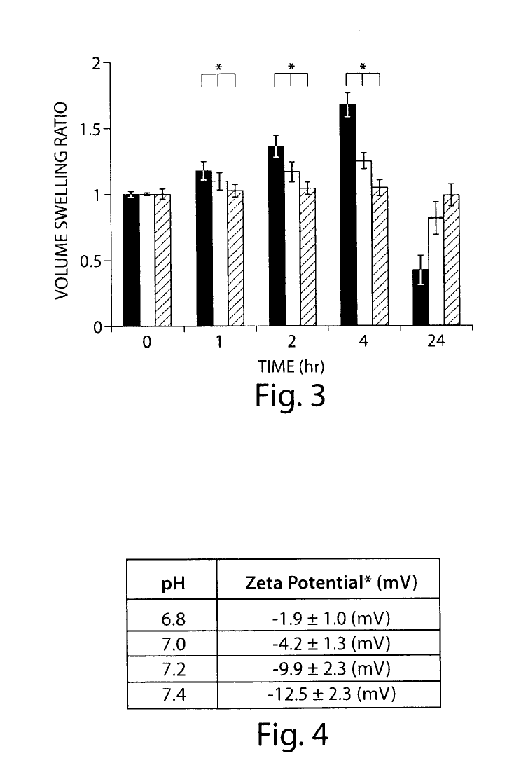

To determine the swelling ratio as a function of crosslinking agent concentration, DMAEMA/HEMA nanoparticles (molar ratio of 10/90) were prepared with 3, 5, and 10 mol % TEGDMA. Swelling studies were performed in a pH 6.8 buffered solution to obtain maximal swelling. FIG. 3 shows the volume swelling ratios of 10/90 (mol/mol) DMAEMA/HEMA nanoparticles crosslinked with 3 (black), 5 (white), or 10 (diagonal) mol % TEGDMA at pH 6.8. As shown in this FIG., the volume swelling ratio of 10/90 DMAEMA/HEMA nanoparticles decreased with increased crosslinking density. The error bars shown correlate to the standard deviation of the mean, where n=3. The statistical significance was calculated relative to 3 mol % at a specific time point, p<0.01.

After 4 hours, the volume swelling ratio at pH 6.8 was 1.7, 1.2 and 1.0 for 3, 5 and 10 mol % TEGDMA, respectively. The swelling rate (and thus sensitivity to pH) of 10/90 DMAEMA/HEMA nanoparticles decreased from 40 nm/hr at 3 mol % TEGDMA to 15 nm/hr for 5 mol % TEGDMA. Nanoparticle degradation may also be influenced by the crosslinking density. The average diameter of 10/90 DMAEMA/HEMA nanoparticles remained unchanged after 24 hours when crosslinked with 10 mol % TEGDMA. Here, the increase in crosslinking density may slow due to hydrolysis-mediated degradation.

The equilibrium swelling volume is a balance between the osmotic pressure of the protonated polymer network and the elasticity of the network. Consequently, the amount of DMAEMA incorporated may affect the degree of pH sensitivity due to the content of quaternizable tertiary amine groups. Therefore, increasing the crosslinking density may reduce the matrix elasticity and nanoparticle degradation.

To determine the effective surface charge of 10/90 DMAEMA/HEMA nanoparticles, the zeta potential was measured at four different pH values (pH 6.8, 7.0, 7.2 and 7.4) as shown in the table given in FIG. 4. The zeta potential is an abbreviation for electrokinetic potential in colloidal systems. The zeta potential of the nanoparticles were consistently negative between pH 6.8 and 7.4. The zeta potential was −12.4±2.3 mV at pH 7.4 and approaches zero at pH 6.8 (−1.9±1.0 mV). The strong negative charge may arise from deprotonated hydroxyl groups of HEMA (—O⁻). As the pH decreases, a proportion of the hydroxyl groups may become protonated (—OH). In addition, DMAEMA may become positively charged as the pH decreases, therefore reducing the strong effective negative charge. A strong negative charge may stabilize the nanoparticles from aggregation. As the nanoparticles approach pH 6.8, the nanoparticle charge approaches zero. However, no aggregation was observed at pH 6.8.

### Example 4

The following example describes the controlled release of paclitaxel (Sigma, St. Louis, Mo., USA) from DMAEMA/HEMA nanoparticles. The release was carried out in a buffered medium at either pH 6.8, 7.0, 7.2, or 7.4. A predetermined amount of freeze-dried DMAEMA/HEMA nanoparticles were placed into a 20 mL scintillation vial containing 10 mL of an aqueous release medium. During release experiments, the vial was placed in a shaker in an incubator with a shaking rate of 100±1 rpm. Samples were collected from the tube once every hour until 6 hours and then every 6 hours until 24 hours. The volume of each collected sample was 350 μL. To maintain a uniform concentration of paclitaxel in the media, fresh buffer was added after sampling during the release studies. To demonstrate the controlled release of paclitaxel followed by a pH drop, an additional test was designed. Paclitaxel-loaded nanoparticles were added to a pH 7.4 buffered medium. After 2 hours, the pH of the buffered medium was dropped to either pH 6.8, 7.0, or 7.2 by adding 1N HCl. A 350 μL sample was collected once every 30 min until 6 hours. The concentration of released paclitaxel was directly determined by a UV/Visible spectrophotometer (SpectraMax Plus384; Molecular Devices Corp., Sunnyvale, Calif., USA) at the absorption wavelength of 229 nm with standard calibration curves.

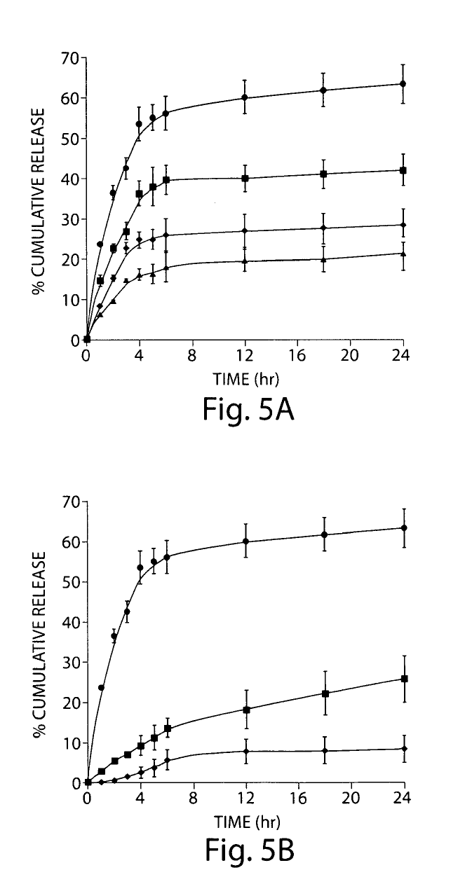

Specifically, the controlled release of paclitaxel from 10/90 DMAEMA/HEMA nanoparticles crosslinked with 3 mol % TEGDMA was studied. This formulation had the largest volume swelling ratio (FIG. 2B) and swelling rate according to the studies described above. As shown in FIG. 5, paclitaxel-loaded 10/90 (mol/mol) DMAEMA/HEMA nanoparticles crosslinked with 3 mol % TEGDMA were monitored for paclitaxel release in media at pH 6.8 (circles), 7.0 (squares), 7.2 (diamonds), and 7.4 (triangles) as shown in FIG. 5A or at pH 6.8 but crosslinked with 3 (circles), 5 (squares), or 10 (diamonds) mol % TEGDMA as shown in FIG. 5B. The error bars shown correlate to the standard deviation of the mean, where n=3. The encapsulation efficiency of the nanoparticles was 76±3%, where drug loss occurs during subsequent washing of the nanoparticles.

The release of paclitaxel may be described by different initial slopes, reaching an asymptote after approximately 4 hours. The volume swelling ratio coincidently reaches a maximum after 4 hours, which may demonstrate the relationship between swelling and release. Large swelling (Q=1.7 in 4 h, at pH 6.8) resulted in a large release (54%, 4 h); whereas small swelling (Q=1.1 in 4 h, at pH 7.2) resulted in slow release (13%, 4 h). Thus, the design of nanoparticles with a large swelling ratio may coincide with fast release.

Generally, the drug release rate may depend on several factors, including: the polymer structure, concentration of polymer and crosslinking agent, the volume swelling ratio, and the interaction between polymer and drug. Among these factors, the volume swelling ratio may be a good indicator of the ability of paclitaxel to be released. The swelling of the nanoparticle structure at a lower pH may lead to an increase in the network mesh size. Changes in the network structure may allow paclitaxel to diffuse out of the matrix within a short time compared to the compact network structure at pH 7.4.

FIG. 5B shows that paclitaxel release may be dependent on the crosslinking agent concentration. At low crosslinking density (3 mol % TEGDMA), paclitaxel was released quickly relative to nanoparticles prepared with 5 and 10 mol % TEGDMA. Increasing the TEGDMA concentration resulted in a higher degree of crosslinking and a reduction in mesh size. Paclitaxel release was reduced by 67% by increasing the TEGDMA concentration from 3 to 5 mol %. Thus, paclitaxel release may be optimized by varying the DMAEMA content, extent of crosslinking, and the pH of the environment.

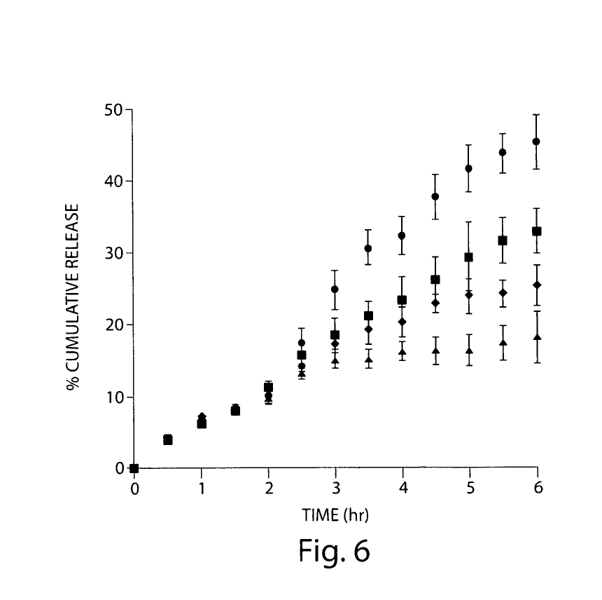

The triggered paclitaxel release was observed by incubating 10/90 (mol/mol) DMAEMA/HEMA nanoparticles crosslinked with 3 mol % TEGDMA for 2 hours at pH 7.4 (triangles) followed by a reduction in pH to either 6.8 (circles), 7.0 (squares), or 7.2 (diamonds) for 4 hours as shown in FIG. 6. The error bars correspond to the standard deviation of the mean, where n=3. Here, 10/90 DMAEMA/HEMA nanoparticles prepared with 3 mol % TEGDMA were allowed to release paclitaxel in pH 7.4 buffered media. After 2 hours, the pH was shifted downwards to either pH 6.8, 7.0, or 7.2 relative to the control (pH 7.4). As shown in FIG. 6, the release of paclitaxel from the nanoparticles was identical until the pH is changed. After the drop in pH, the release varies with pH.

### Example 5

The following example gives results regarding cell viability assays. To determine the toxicity profile of the newly investigated DMAEMA/HEMA nanoparticles, a Live/Dead (viability/cytotoxicity) assay for mammalian cells (Molecular Probes, Eugene, Oreg., USA) was used with HeLa cells (CCL-2, ATCC, Rockville, Md., USA). HeLa cells (10⁴ cells/well) were added to a 96-well cell culture dish (Falcon) with 100 μL of Dulbecco's Modified Eagles Medium (DMEM) supplemented with 5% fetal bovine serum (FBS) and 1% penicillin-streptomycin. All cell culture media and reagents, unless otherwise mentioned, were purchased from Invitrogen (Carlsbad, Calif., USA). After seeding the cells for 24 h, DMAEMA/HEMA nanoparticles were resuspended with growth medium and added into the cell culture dish. After a 24-h incubation, HeLa cells were rinsed twice with 100 μL of Dulbecco's phosphate-buffered saline (D-PBS). The Live/Dead assay reagent, which contained 4 mM calcein AM and 2 mM Ethidium homodimer-1, was added to the cell culture dish. After a 30 min incubation at room temperature, cell viability/cytotoxicity was measured with a fluorescence microplate reader (SpectraMax Gemini XPS; Molecular Devices Corp., Sunnyvale, Calif., USA). All cultures were performed at 37° C., balanced with 5% CO₂ in air in a humidified incubator.

For delivery to tumors, the intravascular administration of paclitaxel-loaded nanoparticles in a pH 7.4 environment was investigated. At pH 7.4, a minimal release of the chemotherapeutic drug is preferred. In the vicinity of the tumor microenvironment, which may have a lower pH, the DMAEMA/HEMA nanoparticle would then release its payload. This would alter the biodistribution of paclitaxel which may result in a decrease in side effects.

After delivery of paclitaxel-loaded nanoparticles, approximately 10% of the paclitaxel was released within the first 2 hours at pH 7.4. As time progresses, the release is dependent on the microenvironment of the nanoparticles. If the microenvironment remains at pH 7.4, then 16% of the paclitaxel will be released over the 6 hour period. However, if the pH of the environment decreases (e.g. drops to 7.2 or 7.0) then release of paclitaxel over 6 hours increases to 22 and 30%, respectively. In the most dramatic case, when the pH drops to 6.8, then 45% paclitaxel will be released over a 6 hour period. Thus, DMAEMA/HEMA nanoparticles may be used to control the rate of release and to trigger release within a local environment.

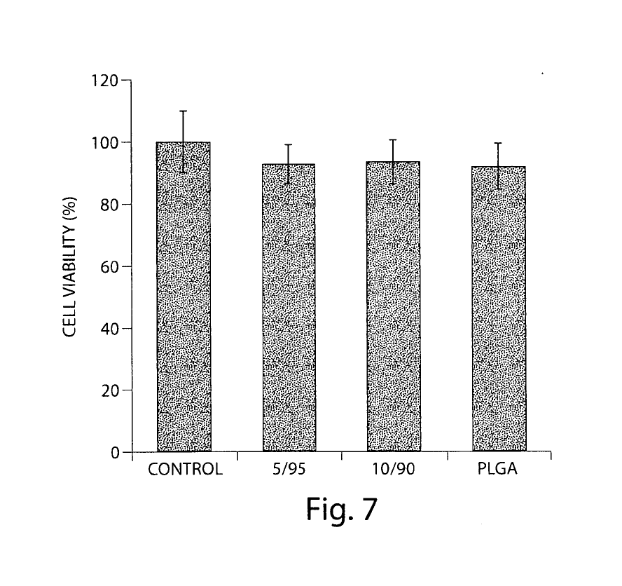

FIG. 7 demonstrates that these carriers are biocompatible. As discussed above, 5/95 and 10/90 (mol/mol) DMAEMA/HEMA nanoparticles crosslinked with 3 mol % TEGDMA were incubated with HeLa cells for 24 hours and compared to a control (without nanoparticles) for viable cell number. Poly(lactic-co-glycolic acid) (PLGA) nanoparticles (average diameter 169.4±11.8 nm) were also tested for cell viability for comparison of DMAEMA/HEMA nanoparticles. The error bars shown correlate with the standard deviation from the mean, where n=3. DMAEMA/HEMA nanoparticles may be used for tumorigenic cancer therapies but may also be used as a nano-scaled pressure sensor as described. In addition, pH-sensitive nanoparticles may be useful in the treatment of ischemia, diabetic ketoacidosis, and in morphine overdoses.

### Example 6

When the nanoparticles are employed for gene therapy, the following conditions may be advantageous. 0.2 g/mL ammonium persulfate and 0.75 g/mL sodium metabisulfite may be employed as the initiator. The system may not require heating. Room temperature or about 5-10° C. may be substantial for the polymerization reaction to proceed. These conditions can lead to narrower nanoparticle size distribution with these initiators. The DMAEMA/HEMA particles synthesized using these conditions were used to investigate the particles use for gene therapy.

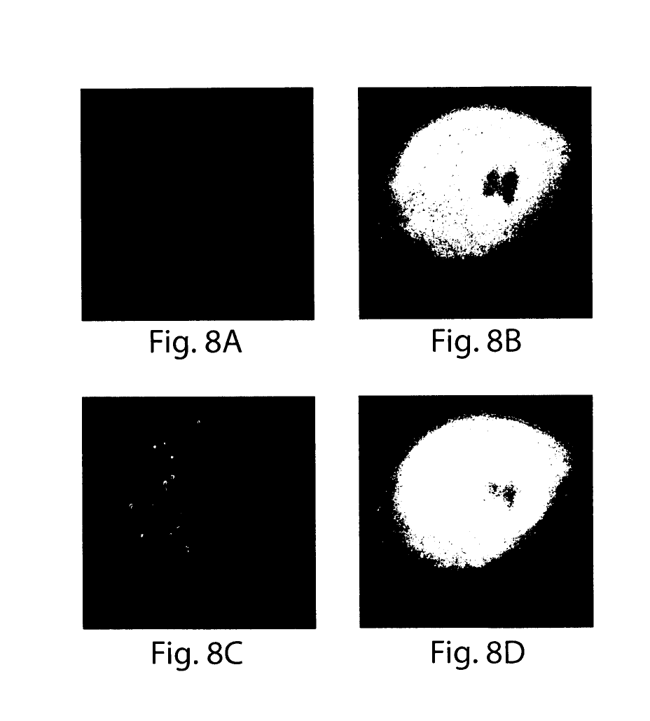

Green fluorescence protein (GFP) plasmid was encapsulated in the DMAEMA/HEMA particles The DMAEMA/HEMA particles were also labeled with quantum dots so the particles could be viewed by fluorescence. The particles were incubated for 24 hours with HeLa cells. FIG. 8 shows DMAEMA/HEMA particles labeled with quantum dot, green fluorescence protein (GFP) expressed due to delivery of DNA. The nucleus was stained with Hoescht. The scale bar correlates to 10 μm. FIG. 8A shows the nucleus. FIG. 8B shows the GFP expressed. FIG. 8C shows the quantum dots attached to the particles. FIG. 8D is an overlay of FIGS. 8A-8C.

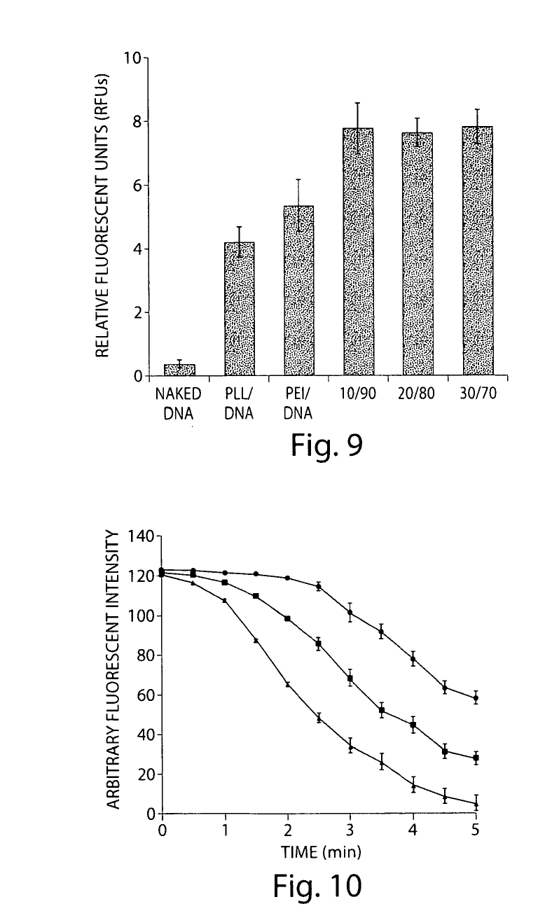

The experiment as given above was conducted using various ratios of DMAEMA/HEMA. FIG. 9 shows a graph of gene transfection versus various DMAEMA/HEMA formulations. As shown by the figure, an increase in the DMAEMA/HEMA ratio gives rise to higher gene transfection. The graph also shows control date obtained using polyethyleneimine (PEI) and polylysine (PLL).

FIG. 10 is a graph of the fluorescence loss in photobleaching (FLIP). This data helps determines the diffusion rate of a 20,000 Da FITC labeled Dextran diffusing through the DMAEMA/HEMA matrix. The FLIP of 30/70 (mol/mol) DMAEMA/HEMA matrix encapsulating FITC-dextran (MW 20,000) at different pH is shown (5.5, triangles; 6.5, squares; 7.4, circles). The diffusion increased as the pH was decreased.

### Example 7

The following describes a similar example of the synthesis and characterization of nanoparticles for drug delivery. This example reports the swelling ratio, zeta potential, diffusivity, matrix elasticity, etc., which are dependent on the monomer ratio, crosslinking density, etc., as described herein.

DNA encoding for green fluorescent protein (GFP) was encapsulated within a series of pH-sensitive nanoparticles comprised of dimethylaminoethyl methacrylate (DMAEMA, a pH-sensitive monomer), 2-hydroxyethyl methacrylate (HEMA, a nonionic monomer), and tetraethylene glycol dimethacrylate (TEGDMA, a crosslinker). DMAEMA has a quaternizable tertiary amine that increased the cationic character of the matrix below its pKₐ of 7.5, and resulted in electrostatic repulsion of ions in solution and volumetric swelling. The DMAEMA/HEMA ratio (10/90, 20/80, or 30/70, mol/mol) and the concentration of TEGDMA crosslinker (3, 6, or 9 mol % TEGDMA) was varied to assess particles with a range of physical properties.

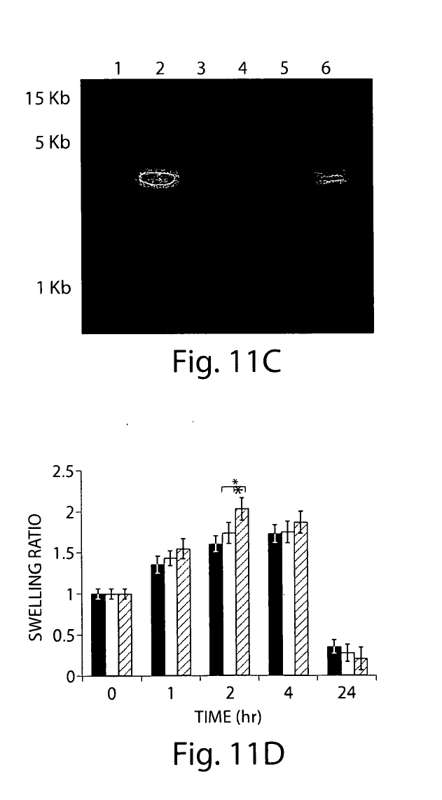

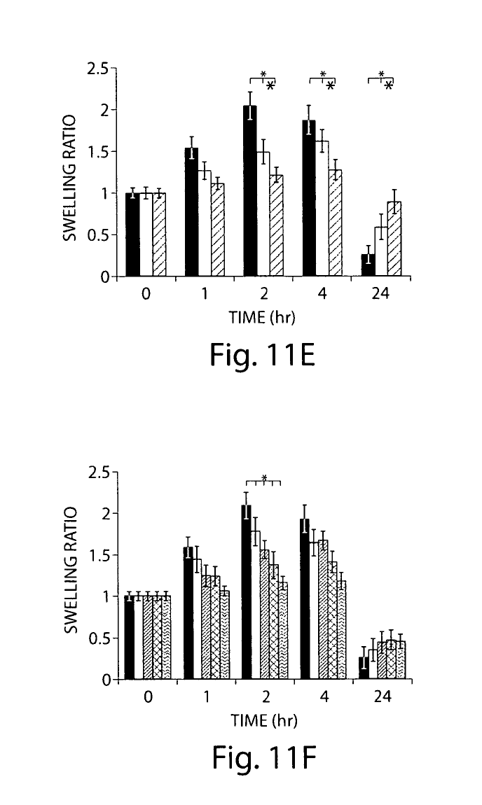

DMAEMA/HEMA nanoparticles were prepared by an oil-in-water (O/W) emulsion. The average size (Table 1) and morphology (FIG. 11A) of nanoparticles were similar across the different DMAEMA/HEMA molar ratios and TEGDMA concentrations after synthesis. More specifically, FIG. 11A shows a transmission electron microscopy image of 30/70 (mol/mol) DMAEMA/HEMA nanoparticles crosslinked with 3 mol % TEGDMA. The average particle size was 200±21 nm in diameter. Scale bar=500 nm. DMAEMA/HEMA nanoparticles were spherical and had a smooth surface morphology. Particles were on average 200±21 nm in diameter and were nontoxic (FIG. 11B). FIG. 11B shows cell viability assays. 10/90, 20/80, and 30/70 (mol/mol) DMAEMA/HEMA nanoparticles were crosslinked with 3 mol % TEGDMA were incubated with HeLa cells for 24 h and compared to a control without nanoparticles. The error is the standard deviation from the mean, where n=3. The size range accommodated systemic administration and passive delivery to tumors and sites of inflammation by the enhanced permeability and retention effect, as described herein.

TABLE 1
Particle size and zeta potential analysis of 10/90,
20/80, and 30/70 (mol/mol) DNA-free DMAEMA/HEMA nanoparticles
crosslinked with 3, 6, or 9 mol % TEGDMA as well as
naked DNA, PLL/DNA, and PEI/DNA complexes.

| | Diameter * (nm) | ζ Potential * (mV) |
|---|---|---|
| Naked DNA | 57.6 ± 9.6 | −8.9 ± 1.8 |
| PLL/DNA | 97.2 ± 12.3 | 18.1 ± 2.6 |
| PEI/DNA | 121.3 ± 10.1 | 24.8 ± 5.2 |
| 10/90 ^{a} | 197.2 ± 10.4 | −18.6 ± 2.4 |
| 20/80 ^{a} | 201.8 ± 12.2 | −13.5 ± 1.6 |
| 30/70 ^{a} | 203.4 ± 11.4 | −10.3 ± 0.8 |
| 10/90 ^{b} | 196.5 ± 10.2 | −16.6 ± 1.4 |
| 20/80 ^{b} | 197.6 ± 11.4 | −14.8 ± 2.1 |
| 30/70 ^{b} | 201.2 ± 13.6 | −10.4 ± 2.6 |
| 10/90 ^{c} | 195.4 ± 12.1 | −14.6 ± 0.8 |
| 20/80 ^{c} | 196.8 ± 14.1 | −11.2 ± 1.6 |
| 30/70 ^{c} | 200.2 ± 12.6 | −8.7 ± 0.7 |

In Table 1, (a) Crosslinked with 3 mol % TEGDMA; (b) Crosslinked with 6 mol % TEGDMA; (c) Crosslinked with 9 mol % TEGDMA; and (d) Data were performed in triplicate.

The structural integrity of the GFP-encoding plasmid DNA (pDNA) encapsulated in DMAEMA/HEMA nanoparticles was examined by agarose gel electrophoresis (FIG. 11C). The extracted pDNA was obtained by degrading DMAEMA/HEMA nanoparticles in pH 5.5 phosphate buffer. Comparison of the naked pDNA (lane 2, FIG. 11C) with encapsulated pDNA (lane 6, FIG. 11C) confirmed that the DNA remains intact. Lanes 3-5, FIG. 11C show no presence of DNA in the supernatant after matrix polymerization. These results indicate that 100% encapsulation of pDNA was achieved and confirmed pDNA stability following DMAEMA/HEMA encapsulation. Specifically, FIG. 11C shows agarose gel electrophoresis for the assessment of plasmid DNA integrity extracted from 30/70 (mol/mol) DMAEMA/HEMA nanoparticles crosslinked with 3 mol % TEGDMA. Lane 1; EZ load 1 kb molecular ladder, lane 2; plasmid DNA, lane 3; supernatant after polymerization, lane 4; supernatant after first washing with pH 7.4 phosphate buffer, lane 5; supernatant after second washing with pH 7.4 phosphate buffer, lane 6; plasmid DNA extracted from nanoparticles.

Swelling of the pH-sensitive DMAEMA/HEMA nanoparticles was observed at three monomer ratios (10/90, 20/80, and 30/70, FIG. 11D), three concentrations of crosslinker (3, 6, and 9 mol % TEGDMA, FIG. 11E) and at five pHs (pH 5.5, 6.0, 6.5, 7.0 and 7.4, FIG. 11F) as a function of time. The swelling ratio is defined as the average diameter of the swollen nanoparticles divided by the average diameter of the nanoparticle at pH 7.4. FIG. 11D shows volume swelling ratios of 10/90 (black), 20/80 (white), and 30/70 (diagonal) (mol/mol) DMAEMA/HEMA nanoparticles crosslinked with 3 mol % TEGDMA at pH 5.5. FIG. 11E shows volume swelling ratios of DMAEMA/HEMA (30/70, mol/mol) nanoparticles crosslinked with 3 (black), 6 (white), and 9 (diagonal) mol % TEGDMA at pH 5.5. FIG. 11F shows DMAEMA/HEMA (30/70, mol/mol) nanoparticles crosslinked with 3 mol % TEGDMA at pH 5.5 (black), 6.0 (white), 6.5 (diagonal), 7.0 (cross-hatched), and 7.4 (wave). For FIGS. 11D-F, the error is the standard deviation of the mean, where n=3. *Statistical significance is calculated relative to pH 7.4 at a specific time point, p<0.01.

In some cases, the DMAEMA content affected the matrix swelling properties. Higher swelling ratios were obtained by increasing the content of DMAEMA within the nanoparticle matrix. The DMAEMA/HEMA (30/70) nanoparticles had a significantly higher swelling ratio (2.0±0.1) relative to the 10/90 (1.6±0.1) and 20/80 (1.7±0.1) formulations after 2 h at pH 5.5. In addition, the rate of swelling increased with DMAEMA content. A maxima in swelling occurred at 2 h for DMAEMA/HEMA (30/70) nanoparticles whereas 10/90 and 20/80 DMAEMA/HEMA formulations achieved a maxima in swelling after 4 h at pH 5.5. Irrespective of the DMAEMA content, degradation at pH 5.5 results in nanoparticles that were 25% of their original diameter after 24 h. Particles with a high DMAEMA content resulted in a fast response and large volumetric swelling. However, DMAEMA content did not vary the time for degradation, within error.

In some embodiments, the physical properties of the matrix may be tailored by increasing the crosslinking density. For example, DMAEMA/HEMA (30/70) nanoparticles were crosslinked with 3, 6, or 9 mol % TEGDMA. Increasing the TEGDMA concentration resulted in a decrease in the swelling ratio and in the time for degradation. Without wishing to be bound by theory, this indicates that matrix structure was an important parameter in controlling pH-responsive swelling. After 2 h of swelling, the swelling ratio at pH 5.5 was 2.1±0.2, 1.5±0.2, and 1.2±0.1 for 3, 6, and 9 mol % TEGDMA, respectively. This corresponded to a volumetric increase between 20 and 110% relative to the initial particle diameter. The response time of highly crosslinked matrices was longer. A maxima in the swelling ratio occurred after 2 h for nanoparticles crosslinked with 3 mol % TEGDMA, whereas particles formulated with 6 and 9 mol % TEGDMA reached a maxima after 4 h. Degradation may be tailored by increasing the crosslinking density. Nanoparticles crosslinked with 9 mol % TEGDMA had a 25% reduction in particle diameter after 24 h compared to a 75% reduction in particle diameter for particles formulated with 3 mol % TEGDMA. A low degree of crosslinking (3 mol % TEGDMA) led to high swelling ratios and fast degradation.

To observe the range of swelling, DMAEMA/HEMA (30/70) nanoparticles crosslinked with 3 mol % TEGDMA were swollen in pH 5.5, 6.0, 6.5, 7.0, and 7.4 phosphate buffers for 24 h. Particles at pH 7.4 exhibited a 20% increase in volume whereas particles at pH 5.5 showed a 110% increase. At 2 h, the swelling ratios of DMAEMA/HEMA nanoparticles at pH 5.5, 6.0, 6.5, 7.0, and 7.4 were 2.1±0.2, 1.8±0.2, 1.6±0.1, 1.4±0.2, and 1.2±0.1, respectively. The swelling ratio was a function of the protonated DMAEMA groups, which was a direct result of the pH of the environment. DMAEMA/HEMA nanoparticles degraded faster at lower pH. After 24 h, the particles at pH 7.4 were twice the size of particles at pH 5.5. After 2 h, the particles in pH 5.5 and 6.0 buffers began to degrade as determined by a maximum in the swelling ratio. Nanoparticles at pH 6.5, 7.0, and 7.4 continued to swell until 4 h. At 24 h, all of the particles were between 25 to 50% of their original diameter due to bulk erosion. The pH of the environment controlled the extent of swelling, the rate of swelling, and the amount of degradation.

Volumetric swelling of the matrix expanded the network of polymer crosslinks allowing molecules to diffuse more quickly. Table 2 describes the diffusivity of a model molecule (FITC-dextran, 20 kDa) within DMAEMA/HEMA matrices using fluorescent recovery after photobleaching (FRAP). Diffusion coefficients were measured from a series of images taken every 30 s over a period of 5 min on a confocal microscope. The diffusivity for formulations was evaluated with increasing DMAEMA content and crosslinking density at three pHs (5.5, 6.5, and 7.4). Diffusion coefficients correlated with volumetric swelling, where high DMAEMA content, low crosslinking density, and low pH resulted in an increase in molecular transport. For example, the diffusion coefficient at low pH (2.4±0.1, pH 5.5) was greater than at high pH (1.4±0.1, pH 7.4) for DMAEMA/HEMA (30/70) hydrogels crosslinked with 3 mol % TEGDMA. Singular matrix or environmental changes were explained by changes in the diffusion coefficient.

TABLE 2
Diffusion coefficients of FITC-dextran (20 kDa) encapsulated
within DMAEMA/HEMA hydrogels crosslinked with TEGDMA
at pH 5.5, 6.5, and 7.4.

| | D × 10⁷* (cm² · s⁻¹) | | | | |
|---|---|---|---|---|---|
| | molar ratio^{a} | | | | |
| | 10/90 | 20/80 | 30/70 | 30/70 | 30/70 |
| | mol %^{b} | | | | |
| pH | 3 | 3 | 3 | 6 | 9 |
| 5.5 | 1.6 ± 0.2 | 1.9 ± 0.2 | 2.4 ± 0.1 | 1.5 ± 0.10 | 0.7 ± 0.06 |
| 6.5 | 1.1 ± 0.1 | 1.3 ± 0.2 | 1.8 ± 0.2 | 1.0 ± 0.12 | 0.5 ± 0.03 |
| 7.4 | 0.7 ± 0.1 | 1.0 ± 0.1 | 1.4 ± 0.1 | 0.6 ± 0.04 | 0.2 ± 0.02 |

In Table 2,
^{a}Molar ratio of DMAEMA/HEMA;
^{b}Mol percentage of TEGDMA; and
(c) Data were performed in triplicate.

Similar diffusion coefficients were measured when multiple variables are changed. The diffusion coefficients for DMAEMA/HEMA (10/90) nanoparticles crosslinked with 3 mol % TEGDMA showed a similar trend to DMAEMA/HEMA (30/70) nanoparticles crosslinked with 6 mol % TEGDMA. The volumetric swelling of these two formulations was similar, achieving a swelling ratio of approximately 1.7 after 4 h (FIGS. 11D-E). In contrast, the DMAEMA/HEMA (30/70) nanoparticles with 9 mol % TEGDMA and the DMAEMA/HEMA (10/90) nanoparticles with 3 mol % TEGDMA have very different swelling ratios although they both have a diffusion coefficient of 0.7 cm²/s; the swelling ratios are 1.7 and 1.2, respectively. Therefore, the rate at which pDNA was released by diffusion may be tailored, in some embodiments, independently from the extent of particle swelling or degradation.

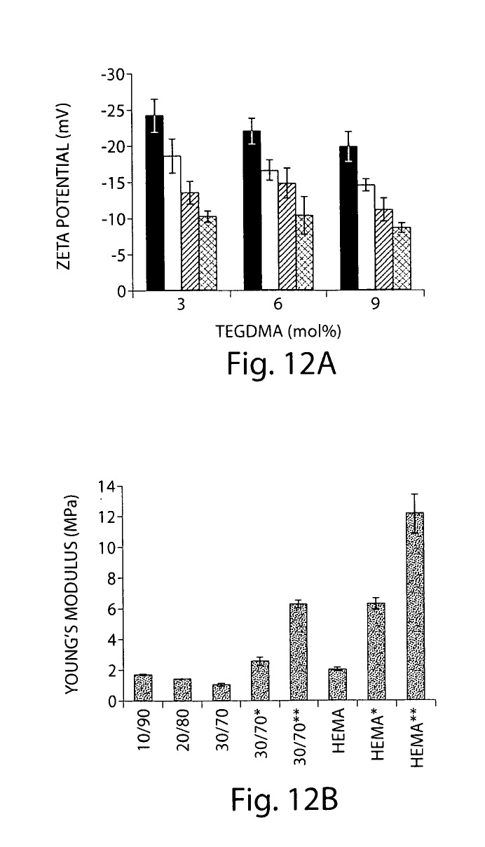

The zeta potential (FIG. 12A) and matrix elasticity (FIG. 12B) of nanoparticles was assessed at pH 7.4 to describe the initial conditions under which gene transfection would occur. Specifically, FIG. 12 shows the comparison of the (A) zeta potential and (B) elastic moduli of different DMAEMA/HEMA hydrogels. For zeta potential measurements, HEMA (100 mol %, black) and DMAEMA/HEMA hydrogels (10/90 (white), 20/80 (diagonal), and 30/70 (cross-hatched), mol/mol) were prepared with 3, 6, or 9 mol % TEGDMA.

All formulations were initially negatively charged, between −8.7 to −18.6 mV. Increasing the DMAEMA content or the mol % of TEGDMA raised the zeta potential (formulations remain negative). Previous work showed that, in some cases, decreasing the pH, and thus protonating the DMAEMA group, increased the zeta potential.

The matrix elasticity did not change significantly with increasing DMAEMA content (from 10/90 to 30/70) however increasing the TEGDMA content resulted in large changes in the Young's modulus. DMAEMA/HEMA (30/70) hydrogels prepared with 3, 6, or 9 mol % TEGDMA had increasing moduli of 1.1±0.1, 2.6±0.3, and 6.3±0.3 MPa, respectively. The DMAEMA/HEMA nanoparticles are elastomeric, similar in magnitude to synthetic rubber.

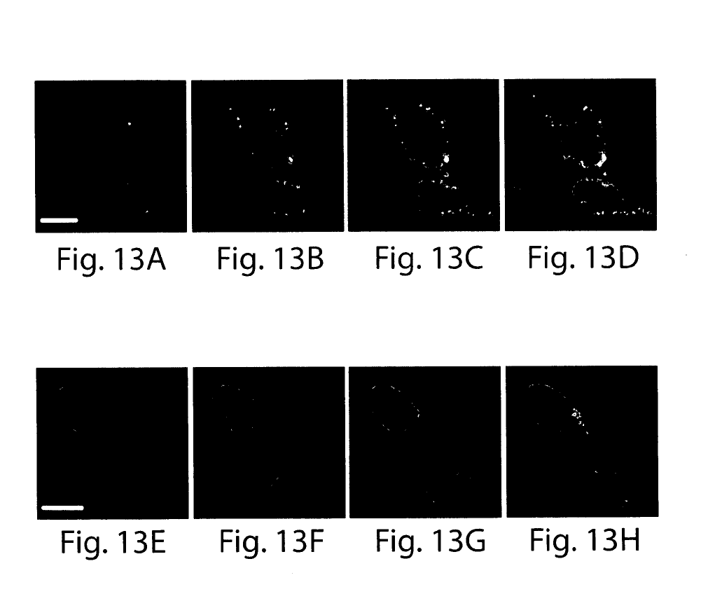

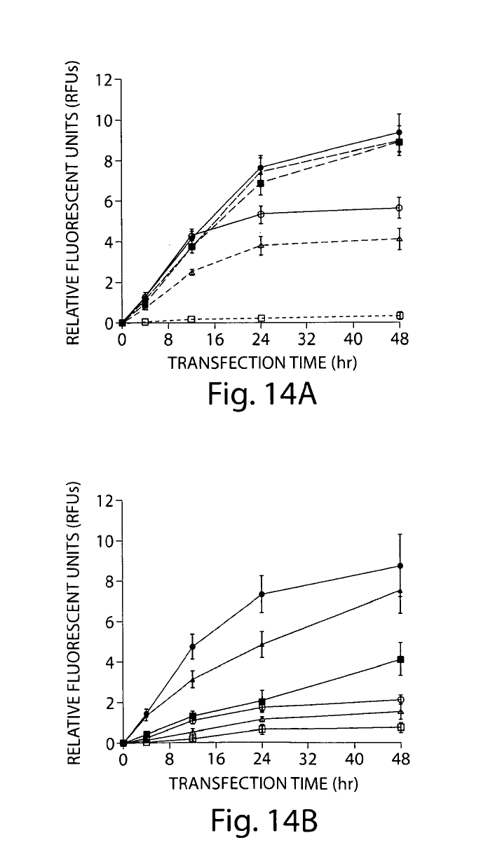

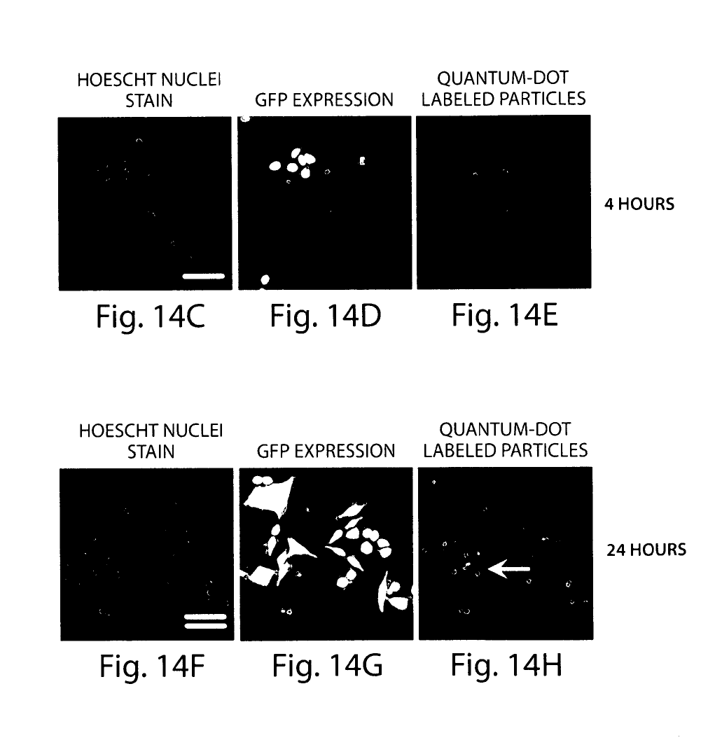

Transfection of HeLa cells by DMAEMA/HEMA nanoparticles was assessed for (1) particle uptake and distribution (FIG. 13), (2) a quantitative analysis of GFP expression as a function of time (FIGS. 14A-B), and (3) a qualitative assessment of GFP expression as a function of time (FIGS. 14C-H). Quantum dot-conjugated DMAEMA/HEMA (30/70) nanoparticles crosslinked with 3 mol % TEGDMA were observed to be taken up more quickly than similar particles crosslinked with 9 mol % TEGDMA. The first appearance of particle uptake was observed within the first hour for 3 mol % TEGDMA but not until 3 h for 9 mol % TEGDMA. At low crosslinking, particles were distributed throughout the cells. This is in contrast to highly crosslinked nanoparticles that have particles localized in the perinuclear region.

More specifically, FIG. 13 shows that quantum dot-conjugated DMAEMA/HEMA (30/70, mol/mol) nanoparticles crosslinked with 3 mol % and 9 mol % TEGDMA were endocytosed by HeLa cells as a function of time (1, 2, 3, and 4 h incubations, respectively). Successive images were taken by confocal microscopy of (A-D) 3 mol % TEGDMA and (E-H) 9 mol % TEGDMA at 1, 2, 3, and 4 h of transfection after addition of quantum dot-conjugated particles. Both scale bar=5 μm.

FIG. 14 relates to the transfection of pH-sensitive particles. FIG. 14A shows HeLa cells which were treated separately with 10/90 (▪), 20/80 (▴), and 30/70 (•) (mol/mol) DMAEMA/HEMA nanoparticles crosslinked with 3 mol % TEGDMA encapsulating pDNA. Transfection with naked DNA (□), PLL/pDNA (Δ), and PEI/pDNA (∘) complexes are used as controls. FIG. 14B shows transfection of HeLa cells treated separately with DMAEMA/HEMA (30/70, mol/mol) nanoparticles crosslinked with 3 (•), 6 (▴), and 9 (▪) mol % TEGDMA encapsulating pDNA and with HEMA nanoparticles crosslinked with 3 (∘), 6 (Δ), and 9 (□) mol % TEGDMA encapsulating pDNA. The error is the standard deviation of the mean, where n=3. FIGS. 14C-H show transfection of HeLa cells treated with DNA encapsulating, quantum dot-conjugated 30/70 (mol/mol) DMAEMA/HEMA nanoparticles crosslinked with 3 mol % TEGDMA. Confocal microscopy was performed at low magnification (20×) after 4 h (FIGS. 14C-E) and 24 h (FIGS. 14F-H) of transfection, respectively. In FIG. 14H, white arrows indicate that quantum dot—conjugated DMAEMA/HEMA nanoparticles are endocytosed into the cellular domain. Scale bar=30 μm.

The extent of transfection was quantified by measuring relative fluorescence units (RFUs) of GFP-expressing HeLa cells at 4, 12, 24, and 48 h. For comparison, the transfection of HeLa cells was performed using naked pDNA, PLL/pDNA complexes, and PEI/pDNA complexes, which are known for their ability to enhance DNA transfection. No differences were observed between pDNA complexes (PLL/pDNA and PEI/pDNA) and pDNA encapsulated within DMAEMA/HEMA nanoparticles at 4 h. At 24 and 48 h, the transfection of DMAEMA/HEMA nanoparticles was twice that of PLL/pDNA complexes and roughly 70% higher than PEUpDNA complexes. GFP transfection was measured at 24 h to yield 6.9±0.6, 7.4±0.7, and 7.7±0.6 RFUs for 10/90, 20/80, and 30/70 DMAEMA/HEMA nanoparticles, respectively. This is in comparison to PLL/pDNA and PEI/pDNA complexes that had measured transfection of 3.8±0.5 and 5.4±0.4 RFUs, respectively. Enhanced DNA transfection was obtained from the use of pDNA encapsulating DMAEMA/HEMA nanoparticles relative to polycation/pDNA complexes and naked pDNA. However, the transfection efficiency was not dependent on the monomer ratio of DMAEMA to HEMA; increasing DMAEMA above 10 mol % did not increase gene transfection.

The effectiveness of DNA delivery was assessed by visual observation of GFP expressing HeLa cells transfected with DMAEMA/HEMA (30/70) nanoparticles with 3 mol % TEGDMA (FIG. 4C-H). The images portray HeLa cells with a nuclei Hoescht stain merged with GFP expression and quantum-dot conjugated nanoparticles. At 4 h, one-third of the cells were expressing GFP which increased to two-thirds at 24 h. Anionic nanoparticles may show increased uptake over cationic polymer/DNA complexes due to size differences. Previous reports indicate that similar uptake between negatively and positively charged nanoparticles have been observed whereas PEG modified (relatively neutral) nanoparticles had reduced uptake.

In some embodiments, matrix elasticity was found to modulate pDNA transfection. An increase in the elastic moduli by altering the TEGDMA concentration resulted in a decrease in pDNA transfection. An increase in TEGDMA content also correlated with a decrease in the molecular diffusivity of the matrix. Similar trends were reported for HEMA and DMAEMA/HEMA (30/70) matrices with increasing TEGDMA content.

Without wishing to be bound by theory, the molecular diffusivity was not the only controlling parameter. Changing the monomer ratio of DMAEMA to HEMA resulted in a range of diffusivities (from 0.7 to 1.4 cm²/s) and volumetric swelling ratios (1.7 to 2.1 at 4 h, pH 5.5); however, increasing the DMAEMA content did not alter the transfection profiles. In contrast, similar elastic moduli (HEMA with 3 mol % TEGDMA vs. DMAEMA/HEMA with 6 mol % TEGDMA or HEMA with 6 mol % TEGDMA vs. DMAEMA/HEMA with 9 mol % TEGDMA) exhibited very different transfection profiles. DMAEMA (for pH sensitivity) and TEGDMA (for matrix elasticity) may be varied to produce nanoparticles with the desired uptake and transfection.

Other pH-sensitive carriers showed advantages in cytosolic delivery. Liposomes incorporating a pH-dependent lipid (dimethylammonium propane, DAP) have been shown to be useful in delivery of short interfering RNA (siRNA). These carriers employed a polycation-b-poly(ethylene glycol) protective layer that was released by an increase in the liposome cationic character upon acidosis. The acidic endosome also mediated liposomal membrane fusion when palmitoylhomocysteine was incorporated into the liposome. Low encapsulation of DNA within liposomes and complicated preparation results in additional expense and time. Core-shell nanoparticles incorporating pH-sensitive diethylaminoethyl methacrylate (DEAEMA) have been used to study cytosolic delivery for immunotherapy. The pH-sensitive core of these particles was cytotoxic and required a nonionic shell.

The DMAEMA/HEMA nanoparticles reported in this example were nontoxic, encapsulated100% DNA, and were prepared using simple procedures. Enhanced transfection was attributed to increased particle uptake, endosomal swelling of DMAEMA/HEMA nanoparticles, and subsequent delivery of the DNA to the cytosol via diffusion. The pH-sensitive DMAEMA/HEMA nanoparticles were efficient gene carriers in comparison with traditional methods which rely on pDNA condensation with PLL or PEI.

### Example 8

The following example outlines the materials and experimental set-up and data relating to Example 7.

Dimethylaminoethyl methacrylate (DMAEMA) and 2-hydroxyethyl methacrylate (HEMA) were purchased from Acros (Morris Plains, N.J., USA). Tetraethylene glycol dimethacrylate (TEGDMA) was obtained from Fluka (St. Louis, Mo., USA). Ammonium persulfate and sodium metabisulphite, the monobasic and dibasic sodium phosphates, N-hydroxysuccinimide (NHS), 1-ethyl-3-(3-dimethylaminopropyl)carbodiimide (EDAC), poly-L-lysine (PLL, MW 26.3 kDa), and polyethyleneimine (PEI, 25 kDa) were purchased from Sigma (St. Louis, Mo., USA). Pluronic F68 was obtained from BASF Corporation (Mount Olive, N.J., USA). Carboxyl quantum dots (655 nm fluorescence emission) and all cell culture media were purchased from Invitrogen (Carlsbad, Calif., USA). All materials were used without further purification. gWiz™ high-expression green fluorescent protein vector (gWiz™ GFP) was purchased from Aldevron (South Fargo, N.D., USA) for gene transfection. Deionized water (18.2 MΩ) was obtained from a Milli-Q purification system (Millipore Corp., Billerica, Mass., USA).

Preparation of DMAEMA/HEMA nanoparticles. An oil-in-water emulsion was prepared by adding 100 μL of a DMAEMA/HEMA solution (at either 10/90, 20/80, or 30/70, mol/mol) to 10 mL of deionized water containing Pluronic F68 (150 mg). For the preparation of DMAEMA/HEMA nanoparticles encapsulating GFP-encoded plasmid DNA, 5 μg of plasmid DNA was directly added to the DMAEMA/HEMA solution and mixed. TEGDMA (3, 6, or 9 mol %) was added to the water phase. When the water phase was homogeneous, aqueous solutions of ammonium persulfate (0.5% w/v) and sodium metabisulphite (0.25% w/v) were added as initiators. The solution was sonicated (200 W, 20 kHz; Digital sonifier 250, Branson Ultrasonics Corp., Danbury, Conn., USA) in a laminar flow hood over an ice bath for 10 min. The polymerization process was carried out at room temperature for 3 h. DMAEMA/HEMA nanoparticles were collected by high-speed centrifugation at 39000×g for 20 min (Sorvall RC26 Plus, SA-600 rotor; Thermo Fisher Scientific Inc., Waltham, Mass., USA). The particles were washed twice with pH 7.4 phosphate buffer to remove residual surfactant and initiators at the aforementioned operating conditions for the centrifuge. Nanoparticles were freeze-dried (FTS systems, INC., Stone Ridge, N.Y., USA) overnight and stored at 4° C. for later use.

Characterization of DMAEMA/HEMA nanoparticles. To determine the size and morphology of DMAEMA/HEMA nanoparticles by transmission electron microscopy (TEM, JEOL 2100; JEOL Ltd., Tokyo, Japan), a 0.05 wt % aqueous nanoparticle suspension was prepared. One drop of the nanoparticle solution was placed on a copper grid (300 mesh, TED PELLA Inc., Redding, Calif., USA) supporting a thin film of amorphous carbon. The excess liquid was removed with filter paper and the copper grid was dried in the hood. The particle size was analyzed by dynamic light scattering (ZetaPALS; Brookhaven Instrument, Holtsville, N.Y., USA) before and after swelling in different pH buffers. The zeta potential of the nanoparticles was measured by electrophoresis (Zeta PALS; Brookhaven Instrument, Holtsville, N.Y., USA). Nanoparticles were diluted to 0.1 mg/mL in a 10 mM N-[tris(hydroxymethyl)methyl]-2-aminoethanesulfonic acid (TES) buffer at pH 7.4. The zeta potential was measured a minimum of three times.

Swelling studies. Swelling of DNA-free DMAEMA/HEMA nanoparticles was performed in buffered solutions with known pH (either pH 5.5, 6.0, 6.5, 7.0 or 7.4) and composition (10 mM TES, adjusted with 1 N NaOH) at 37±0.5° C. A predetermined amount of freeze-dried DMAEMA/HEMA nanoparticles (10/90, 20/80, or 30/70, mol/mol) crosslinked with TEGDMA (3, 6, or 9 mol %) were suspended in 10 mL of buffer. Samples were placed on a shaker (OS-500 orbital shaker, VWR, West Chester, Pa., USA) at a shaking rate of 100±1 rpm in an incubator maintained at 37° C. The average nanoparticle diameter was measured by dynamic light scattering at 0, 1, 2, 4, and 24 h. The swelling ratio are reported as the initial particle diameter (pH 7.4) divided by the swollen particle diameter (pH 5.5, 6.0, 6.5, or 7.0).

Gel electrophoresis. The integrity of the encapsulated plasmid DNA in DMAEMA/HEMA nanoparticles (30/70 molar ratio, crosslinked with 3 mol % TEGDMA) was analyzed by agarose gel electrophoresis in tris-acetate-EDTA buffer (TAE: 40 mM tris-base, 1 mM EDTA, pH 8.5). 5 μg DNA-loaded nanoparticles (1 mg) was suspended in 500 μL phosphate buffer at pH 5.5. The suspension was vortexed for 1 h to break down the particles. For gel electrophoresis, the water phase was collected after centrifugation at 15000×g for 20 min at 4° C. and loaded onto a 1.0 w/v% agarose gel containing ethidium bromide (0.5 μg/mL). Electrophoresis was performed in TAE buffer at 100 V. The gel was visualized on a UV transilluminator (Model-20, UVP Inc., Upland, Calif., USA) alongside a DNA ladder to indicate the location and size of the DNA.

Quantum dot conjugation with DMAEMA/HEMA nanoparticles. Quantum dot-conjugated nanoparticles were prepared to track particle uptake and distribution. The N-terminal group of the DMAEMA/HEMA nanoparticle was conjugated to the surface of carboxylated quantum dots (655 nm emission) using a carbodiimide reaction. EDAC (0.05 mg) and NHS (0.025 mg) were dissolved in 1 mL 0.1 M MES buffer to which 10 μL of the quantum dot stock solution was added. After 1 hour, 100 μL of DMAEMA/HEMA nanoparticle stock solution was gently added and reacted for 6 hours at room temperature. Quantum dot-conjugated DMAEMA/HEMA nanoparticles were washed twice with pH 7.4 phosphate buffer to remove unreacted quantum dots and residual EDAC and NHS. The labeled nanoparticles were stored at 4° C. for later use. Fluorescence recovery after photobleaching (FRAP). FRAP experiments were performed on a Zeiss inverted confocal microscope using a 10× objective lens. Hydrogels of DMAEMA/HEMA (10/90, 20/80, or 30/70, mol/mol) crosslinked with TEGDMA (3, 6, or 9 mol %) encapsulating fluorescein isothiocyanate-dextran (0.1 wt %, FITC-dextran; MW 20 kDa) were prepared in the shape of a disk. The average diameter and thickness of the DMAEMA/HEMA disks were 200±12.8 μm and 80±3.2 μm respectively. The disks were placed in a 35 mm diameter glass bottom tissue culture dish (MatTek Co., Ashland, Mass., USA) with 5 mL pH buffer (at either pH 5.5, 6.5, or 7.4). The diffusion coefficients were determined by a time series of captured images obtained from the confocal microscope. Samples were bleached for 5 min using 100% power from an argon ion laser. Data were collected as a series of image frames, captured at 30 s intervals. The fluorescent intensity profile was measured by the LSM510 Meta imaging software and Image J (NIH) with the same size and shape of DMAEMA/HEMA hydrogel disks. The diffusion coefficient (D) was measured for different compositions (molar ratios of DMAEMA/HEMA and mol % TEGDMA concentration) and for different pH conditions. The diffusion coefficient was calculated from D=w²/4t_{1/2} (assuming a Gaussian profile for the bleaching beam), where w is the width of the beam and t_{1/2} is the time required for the bleach spot to recover half of its initial intensity.

Gene transfection. HeLa cells in the exponential growth phase were detached with 1× ethylene-diamine tetra-acetic acid (EDTA)-trypsin from a tissue culture dish (Falcon, Frankin Lakes, N.J., USA). HeLa cells (5×10⁴ cells/well) were inoculated in a 96-well plate with 200 μL of high glucose Dulbecco's modified Eagle medium (DMEM) supplemented with 10% fetal bovine serum (FBS),and 1% penicillin-streptomycin at 37° C. in a 5% CO², humidified incubator. After an 18 h incubation, 20 μL of the nanoparticle suspensions (containing 5 μg plasmid) were diluted with serum-free media. Prior to transfection, cell growth media was removed and the cells were washed with PBS three times to remove the serum component. Then, DNA-loaded nanoparticle suspensions were added and cells were cultured for 4 h. The serum-free media was replaced with fresh growth media containing serum and 1% penicillin-streptomycin. The fluorescence intensity of transfected HeLa cells was measured at 4, 12, 24, and 48 h after the addition of DNA-loaded nanoparticles relative to non-transfected HeLa cells. The transfection efficiency was analyzed by relative fluorescent units (RFUs) using a fluorescent microplate reader (SpectraMax Gemini XPS; Molecular Devices Corp., Sunnyvale, Calif., USA). For comparison, transfection of HeLa cells was performed using equivalent amounts of DNA by administration of either naked DNA, PLL complexed with DNA (MW of PLL=26.3 kDa, N/P ratio=2, where N is the amount of amino nitrogens and P stands for the amount of phosphate groups of the plasmid DNA), or PEI complexed with DNA (MW of PLL=750 kDa, N/P ratio=6).

DNA encapsulating, quantum dot-conjugated DMAEMA/HEMA nanoparticles were observed for uptake and distribution. HeLa cells (5×10⁴) were cultured on 35-mm diameter glass-bottom tissue culture dishes. After an 18 h pre-incubation, the culture medium was replaced with quantum dot-conjugated DMAEMA/HEMA nanoparticles suspended in fresh 2 mL DMEM plus 10% FBS. Cells were washed with D-PBS three times and examined under confocal microscopy at different time points for different nanoparticle formulations. Each population of cells was stained with Hoescht stain to confirm the nucleus.

Cell viability assay. The cytotoxicity of DNA free DMAEMA/HEMA nanoparticles was evaluated by a Live/Dead (viability/cytotoxicity) assay for mammalian cells (Molecular Probes, Eugene, Oreg., USA) with HeLa cells. HeLa cells (10⁴ cells/well) were added to a 96-well cell culture dish (Falcon) with 100 μL DMEM supplemented with 5% FBS and 1% penicillin-streptomycin. After inoculating the cells for 18 h, DMAEMA/HEMA nanoparticles were resuspended with growth medium and added into a cell culture dish. After a 24 h incubation, HeLa cells were rinsed twice with 100 μL of Dulbecco's phosphate-buffered saline (D-PBS). The Live/Dead assay reagent, which contained 4 mM calcein AM and 2 mM Ethidium homodimer-1, was added to the cell culture dish. After a 30 min incubation at room temperature, cell viability/cytotoxicity was measured with a fluorescence microplate reader (SpectraMax Gemini XPS; Molecular Devices Corp., Sunnyvale, Calif., USA). All cultures were performed at 37° C., balanced with 5% CO₂ in air in a humidified incubator.

Elastic Modulus of DMAEMA/HEMA gels. The elastic moduli of 10/90, 20/80, and 30/70 (mol/mol) DMAEMA/HEMA hydrogels crosslinked with 3 mol % TEGDMA were measured after polymerization. For comparison, 100 mol % HEMA and 30/70 (mol/mol) DMAEMA/HEMA hydrogels were also crosslinked with different concentrations of TEGDMA (3, 6, and 9 mol %, respectively). Stress as a function of strain was measured under tensile conditions for each hydrogel (30 mm×10 mm×2 mm) on an Instron 5543 (Instron, Canton, Mass., USA) using a 500 N loading cell. Hydrogels were strained to failure at a rate of 2 mm/min. The elastic modulus was calculated from the initial 40% strain. Three hydrogel samples of each formulation were tested.

Statistical analysis. All of the experimental data were obtained in triplicate unless otherwise mentioned and are presented as mean±standard deviation. Statistical comparison by analysis of variance was done at a significance level of p<0.01 based on the Student's t-test.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A method for delivering a therapeutic or diagnostic agent, comprising:
providing a pH-responsive nanostructure comprising a polymeric material and a therapeutic or diagnostic agent;
exposing the pH-responsive nanostructure to a first set of conditions, wherein the pH-responsive nanostructure releases the therapeutic or diagnostic agent at a first release rate; and
exposing the pH-responsive nanostructure to a second set of conditions, wherein the pH-responsive nanostructure releases the therapeutic or diagnostic agent at a second release rate that is at least 20% greater than the first release rate,
wherein the first and second sets of conditions comprise different pH values.

2. A method for delivering a therapeutic or diagnostic agent, comprising:
providing a pH-responsive composition comprising a therapeutic or diagnostic agent;
exposing the pH-responsive composition to a first set of conditions, wherein the pH-responsive composition releases the therapeutic or diagnostic agent at a first release rate; and
exposing the pH-responsive composition to a second set of conditions, wherein the pH-responsive composition releases the therapeutic or diagnostic agent at a second release rate that is at least 20% greater than the first release rate,
wherein the first pH value differs from the second pH value by less than 1.0 pH unit.

3. A method for delivering a therapeutic or diagnostic agent, comprising:
providing a pH-responsive composition comprising a therapeutic or diagnostic agent;
exposing the pH-responsive composition to a first set of conditions, wherein the pH-responsive composition releases the therapeutic or diagnostic agent at a first release rate; and
exposing the pH-responsive composition to a second set of conditions, wherein the pH-responsive composition releases the therapeutic or diagnostic agent at a second release rate that is at least 20% greater than the first release rate,
wherein the first pH value is greater than the second pH value.

4. A method as in claim 1, wherein the therapeutic or diagnostic agent is delivered to a mammal.

5. A method as in claim 4, wherein the mammal is a human.

6. A method as in claim 1, wherein the first pH value differs from the second pH value by less than 1.0 pH unit.

7. A method as in claim 1, wherein the first pH value differs from the second pH value by less than 0.5 pH unit.

8. A method as in claim 1, wherein the first pH value differs from the second pH value by less than 0.2 pH unit.

9. A method as in claim 1, wherein the first pH value differs from the second pH value by less than 0.1 pH unit.

10. A method as in claim 1, wherein the nanostructure is a particle having an average particle size of less than 500 nm.

11. A method as in claim 1, wherein the nanostructure is a particle having an average particle size of less than 400 nm.

12. A method as in claim 1, wherein the nanostructure is a particle having an average particle size of less than 300 nm.

13. A method as in claim 1, wherein the nanostructure is a particle having an average particle size between about 100 nm and about 300 nm.

14. A method as in claim 1, wherein the nanostructure is a particle having an average particle size between about 100 nm and about 250 nm.

15. A method as in claim 1, wherein the nanostructure is a particle having an average particle size of less than about 100 nm.

16. A method as in claim 1, wherein the release rate of the pH-responsive nanostructure is not affected by a change in temperature.

17. A method as in claim 1, wherein the pH-responsive nanostructure releases the therapeutic or diagnostic agent upon swelling.

18. A method as in claim 17, wherein the swelling of the nanostructure comprises a volumetric change of at least 0.1%

19. A method as in claim 17, wherein the swelling of the nanostructure comprises a volumetric change of at least 0.5%.

20. A method as in claim 17, wherein the swelling of the nanostructure comprises a volumetric change of at least 1%.

21. A method as in claim 17, wherein the swelling of the nanostructure comprises a volumetric change of at least 5%.

22. A method as in claim 1, wherein the first pH value is about 7.4.

23. A method as in claim 1, wherein the second pH value is less than 7.4.

24. A method as in claim 1, wherein the second pH value is less than 7.2.

25. A method as in claim 1, wherein the second pH value is less than 7.0.

26. A method as in claim 1, wherein the second pH value is less than 6.8.

27. A composition for delivery of a therapeutic or diagnostic agent, comprising:
a pH-responsive nanostructure comprising a polymeric material, wherein the polymeric material comprises hydrophilic repeating units and pH-responsive repeating units, the ratio of hydrophilic repeating units to pH-responsive repeating units being at least 1.0; and
a therapeutic or diagnostic agent associated with the polymeric material,
wherein the pH-responsive nanostructure has an average particle size of less than 500 nm.

28. A composition for delivery of a therapeutic or diagnostic agent, comprising:
a pH-responsive nanostructure comprising a polymeric material, wherein the polymeric material comprises hydrophilic repeating units and pH-responsive repeating units, the ratio of hydrophilic repeating units to pH-responsive repeating units being at least 1.0; and
a therapeutic or diagnostic agent associated with the polymeric material,
wherein, when exposed to a decrease in pH of 1.0 pH unit or less, the pH-responsive nanostructure undergoes an increase in volume.

29. A composition for delivery of a therapeutic or diagnostic agent, comprising:
a plurality of pH-responsive nanostructures each comprising a base material and a therapeutic or diagnostic agent associated with the base material,
wherein the therapeutic or diagnostic agent has a first release rate under a first set of physiologically-compatible conditions at a first pH, and a second release rate at least 20% greater than the first rate under a second set of physiologically-compatible conditions at a second pH differing from the first pH by less than 1.0 pH unit.

30. A composition claim as in claim 27, wherein the hydrophilic repeating unit is hydroxyethyl methacrylate.

31. A composition claim as in claim 27, wherein the pH-responsive repeating unit is 2-(dimethylamino)ethyl methyacrylate.

32. A composition as in claim 27, wherein the polymeric material further comprises a crosslinking agent.

33. A composition as in claim 32, wherein the crosslinking agent is tetraethylene glycol dimethacrylate.

34. A composition as in claim 32, wherein the crosslinking agent is present in at least about 0.1 mol % of the total repeating unit amount.

35. A composition as in claim 32, wherein the crosslinking agent is present in at least about 1.0 mol % of the total repeating unit amount.

36. A composition as in claim 32, wherein the crosslinking agent is present in at least about 3.0 mol % of the total repeating unit amount.

37. A composition as in claim 32, wherein the crosslinking agent is present in at least about 5.0 mol % of the total repeating unit amount.

38. A composition as in claim 32, wherein the crosslinking agent is present in at least about 10.0 mol % of the total repeating unit amount.

39. A composition as in claim 27, wherein the polymeric material is formed as nanoparticles.

40. A composition as in claim 39, wherein the therapeutic or diagnostic agent is encapsulated in the nanoparticles.

41. A composition as in claim 27, wherein the therapeutic agent is paclitaxel.

42. A composition as in claim 27, wherein the ratio of hydrophilic repeating units to pH-responsive repeating units is at least 2.0.

43. A composition in claim 27, wherein the ratio of hydrophilic repeating units to pH-responsive repeating units is at least 3.0.

44. A composition as in claim 27, wherein the ratio of hydrophilic repeating units to pH-responsive repeating units is at least 5.0.

45. A composition as in claim 27, wherein the ratio of hydrophilic repeating units to pH-responsive repeating units is at least 10.0.

46. A composition as in claim 27, wherein the ratio of hydrophilic repeating units to pH-responsive repeating units is at least 20.0.

47. A composition as in claim 27, wherein the pH-responsive nanostructure has an average particle size of less than 400 nm.

48. A composition as in claim 27, wherein the pH-responsive nanostructure has an average particle size of less than 300 nm.

49. A composition as in claim 27, wherein the pH-responsive nanostructure has an average particle size of less than 200 nm.

50. A composition as in claim 27, wherein the pH-responsive nanostructure has an average particle size of less than 100 nm.

51. A composition as in claim 27, wherein the pH-responsive nanostructure has an average particle size between about 100 nm and about 300 nm.

52. A composition as in claim 28, wherein the pH-responsive nanostructure is exposed to a decrease in pH of 0.5 pH units or less.

53. A composition as in claim 28, wherein the pH-responsive nanostructure is exposed to a decrease in pH of 0.2 pH units or less.

54. A composition as in claim 28, wherein the pH-responsive nanostructure is exposed to a decrease in pH of 0.1 pH units or less.

55. A composition as in claim 28, wherein the pH-responsive nanostructure is exposed to a decrease in pH of 0.05 pH units or less.
